(19) United States
(12) Patent Application Publication
(10) Pub. No.: US 2022/0387995 A1
(43) Pub. Date: Dec. 8, 2022

(54) An Integrated Smart Point-Of-Care Biosensor for Whole-Blood Liquid Biopsies

(71) Applicant: THE REGENTS OF THE UNIVERSITY OF MICHIGAN, Ann Arbor, MI (US)

(72) Inventors: Katsuo Kurabayashi, Ann Arbor, MI (US); Kevin R. Ward, Superior Township, MI (US); Young Geun Park, Ann Arbor, MI (US); Xiaogan Liang, Ann Arbor, MI (US); Byunghoon Ryu, Ann Arbor, MI (US)

(21) Appl. No.: 17/771,377

(22) PCT Filed: Oct. 26, 2020
(86) PCT No.: PCT/US20/57398

§ 371 Date: Apr. 22, 2022

Related U.S. Application Data
(60) Provisional application No. 62/983,069, filed on Feb. 28, 2020.
(60) Provisional application No. 62/925,369, filed on Oct. 24, 2019.

(51) Int. Cl. B01L 3/00; G01N 21/552; G01N 33/543

(52) U.S. Cl. CPC B01L 3/502715; B01L 3/502761; G01N 21/554; G01N 33/54366; B01L 2300/021; B01L 2300/023; B01L 2300/0636; B01L 2300/0816; B01L 2300/0896; B01L 2400/0478

(57) ABSTRACT

A blood biomarker analysis systems providing fast biomarker identification includes a multimodal bioassay device having a biosensor within a portable pipette-shaped device and using nanoplasmonic barcode detectors, such as formed of antibody conjugated gold nanoparticle arrays (AuNPs), capable of capturing any of a plurality of biomarkers. The biomarker analysis system further includes the pipette-shaped device being smartphone-connected and portable to form a highly accurate, point-of-care bioassay device.

### CROSS-REFERENCE TO RELATED APPLICATION

This application claims benefit of the filing date of U.S. provisional patent application No. 62/925,369, filed Oct. 24, 2019 and U.S. provisional patent application No. 62/983,069, filed Feb. 28, 2020, which provisional applications are both hereby incorporated by reference in their entirety.

### STATEMENT OF GOVERNMENT SUPPORT

This invention was made with government support under ECCS1708706 awarded by the National Science Foundation. The government has certain rights in the invention.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Systemic pro-inflammatory illnesses, such as sepsis, systemic inflammatory response syndrome (SIRS), acute respiratory distress syndrome (ARDS), cytokine release syndrome (CRS), resulting from infection, trauma, burns, surgery, cancer immune therapy, and severe allergy pose serious threats to human health leading to organ dysfunction, organ failure, or mortality. The highly complex and dynamic nature of the immune system experiencing acute inflammation makes it challenging to provide timely precision medicine based therapies. Challenge also exists in providing precision therapies for chronic inflammatory diseases such as inflammatory bowel disease, arthritis, and others. Early identification, trajectory tracking, and precision-health approaches are critical for treating systemic inflammatory illnesses. There is a need for precision diagnosis and treatment of life-threatening and chronic pro-inflammatory disorders at the point of care or injury. Inflammatory and other biologic biomarkers associated with inflammation could greatly aid diagnosis and treatment, but no robust technology to measure such markers exists. In recent years, research efforts have been made for developing point-of-care diagnosis technologies. But existing systems are still premature with limited performance and yet to be implemented in systemic illness analysis outside a hospital setting.

### SUMMARY OF THE INVENTION

The evolution of biomarker-guided precision-medicine therapies targeting specific pathological processes has advanced rapidly, based on a greater understanding of genomic, molecular, and cellular data of an individual patient. With increasing mortality attributed to hundreds of cancer-related diseases, researchers have explored new precision-medicine approaches for these diseases and shown their great promise to guide clinicians in making viable and timely diagnoses and accurate prognoses. In particular, diagnostics guided by prognostic and predictive protein biomarkers in blood offers unprecedented opportunities for discovering cancer at an early stage and continuously monitoring its progression.

Blood-based assays may eliminate cost, time, inconvenience, and invasiveness imposed by conventional cancer screening techniques, such as mammography, colonoscopy, tissue biopsy, radiological imaging, and genetic testing. The early detection of a low-abundant cancer biomarker can significantly reduce cancer mortality and save lives. The conventional “gold standard” methods for such protein biomarker quantification are enzyme-linked immunosorbent assay (ELISA) and bead-based immunoassays, wherein signals are detected by microplate readers. These methods involve sample preparation, incubation with the primary antibody and labeling reagent, along with multiple washing steps; therefore, these methods suffer technological limitations, such as slow detection (˜3-8 h) and consumption of expensive reagents in each assay. Although a large population of people suffers from cancer-related diseases, these methods are not proficient in the frequent monitoring of an individual patient during the treatment, and they require a centralized laboratory involving analytical processes managed by highly trained experts. Therefore, a device allowing the highly-sensitive detection of cancer biomarkers near the patient is essential for continuous cancer monitoring and prognosis, and especially for an early detection.

Point-of-care testing (PoCT) that enables medical/clinical analysis at or near the location of patient care has shown a great potential for precise and personalized health care. PoC systems are to provide fast, cost-effective, and easy-to-use diagnostic testing that shortens the therapeutic turnaround time. Importantly, they are expected to cover patient populations with low socioeconomic status. A potential global market growth from us $23.16 billion in 2016 to us $36.96 billion in 2021, which is estimated based on a compound annual growth rate (CAGR) of 9.8%, truly reflects the future promise of PoCT. Recently, there is a growing interest in PoCT for cancer-related diseases that incorporates nano/biosensors with superior analytical performances and label-free measurement capabilities. However, many of existing biosensors suffer from limited sensitivity. This limits the ability to detect low-abundance (˜pm-level) biomarkers in physiological samples required for diagnosing cancer at a very early stage. Furthermore, achieving high detection accuracy with these biosensors requires high sample purity to suppress background noise due to non-specific binding of blood constituents other than the target biomarker proteins. Therefore, a resource-demanding sample preparation process is needed to isolate purified plasma or serum from whole blood prior to the assay. This process cannot be done by individuals or clinicians at or near the location of the patient, and it poses a major challenge in PoCT using existing biosensors. A biosensor enabling portable, easy-to-operate, high-sensitivity blood protein measurement without sample preparation is imperative for PoCT adoptable in real cancer test.

The present techniques provide novel blood biomarker analysis systems for fast biomarker identification through the use of a multimodal bioassay device capable of operation in the field and health care systems at the point-of-care or near point-of-care. In various examples, biomarker analysis systems include a smartphone-connected, highly portable, pipette-shaped platform device as the bioassay device. In some examples, the biomarker analysis system is trained using machine learning algorithms to detect one or more biomarkers in a very short time window (e.g., in 10 mins or less, in 5 mins or less, or in 1 min or less), with high accuracy.

The biomarker analysis system can therefore be used at the point-of-care, soon after the time of an injury or illness, to allow medical personnel more accurate assessments of a subject's condition and treatment options. The biomarker analysis system may be used at the point-of-care in the intensive care unit (ICU), the general ward, the emergency department, the clinical laboratory, an ambulance, and a remote area under limited resources to detect the early onset and predict outcomes of acute illnesses, such as injury, surgery, sepsis/sepsis shock, asthma, systemic inflammatory response disorder (SIRS), acute respiratory distress syndrome (ARDS), cytokine release syndrome (CRS), and so forth.

In some examples, a smart pipette is provided including a multimodal biomarker sensor and data transmitter. In some examples, the smart pipette includes a wireless data transmitter. In some examples, the smart pipette is part of a biomarker analysis system including a mobile platform, such as a smartphone, that communicates with the smart pipette via the wireless data transmitter to diagnosis biomarker data and generate a biomarker report and/or suggested treatment.

In some examples, the multimodal biomarker sensor is a bioassay implemented in a single channel configuration, where a sample is provided to a single biomarker channel.

In some examples, the multimodal biomarker sensor is a bioassay implemented in a multichannel configuration, where each channel is multiplexed to allow for selectable detection of a different biomarker from the same device. In some examples, the biomarker sensor operates multiple channels simultaneously to allow for multiple different biomarkers to be detected in parallel, for example, at the same time.

In some examples, the biomarker analysis systems include structurally engineered gold nanoparticle biosensor arrays or colloidal nanoparticle biosensors together with atomically thin photoconductive nanosheet channels. These modalities are combined into a highly compact module architecture.

Due to its high-speed, high-sensitivity, and user-friendly operation, the present techniques enable near-real-time bedside monitoring of blood biomarker variations in patients over the course of their systemic illnesses. Patient data can be acquired and transmitted using a smartphone connected with the smart pipette device via Bluetooth. The integration of time-series biomarkers of illness coupled with traditional and nontraditional markers and physiology and organ function, coupled with artificial intelligence techniques like machine learning allows for precision phenotyping and the development of new precision therapies for systemic illnesses and other complex states of inflammation and immune dysfunction.

In an example, a biomarker detector is provided comprising: a sealable housing; an inlet configured to receive fluid; and a sensor device within the sealable housing and communicatively coupled to the inlet to receive the fluid, the sensor further comprising an illumination source, a photodetector array, and a microfluidic chip positioned between the illumination source and the photodetector array, the microfluidic chip comprising a plurality of barcode channels, each barcode channel configured to detect to a different biomarker and each barcode channel configured to affect illumination from the illumination source in response to detection of the respective biomarker, where such affected illumination is detectable by the photodetector array.

In some such examples, a biomarker analysis system having the biomarker detector device further comprises: a mobile computing device external to and configured to wirelessly communication with the biomarker detector device, the mobile computing device having a display and a wireless data transmitter, the mobile computing communicatively coupled to receive biomarker data from the biomarker detector device over a wireless communication link, the mobile computing device having a processor and a memory storing instructions that when executed cause the processor to generate a biomarker report indicating a presence of one or more biomarkers detected by the biomarker detector device and to display the biomarker report on the display of the mobile computing device.

In an example, a biomarker detector device comprises: a sealable housing; an inlet configured to receive fluid; and a biosensor assembly within the sealable housing and communicatively coupled to the inlet to receive the fluid, the biosensor assembly being a multilayered structure comprising an illumination source in a first layer, a photodetector array in a second layer opposing the first layer, and a biosensor in an intermediate layer positioned between the first layer and the second layer, the biosensor being configured with at least one near infrared (NIR) sensitive plasmonic nanoprobe for colorimetric cancer biomarker detection by affecting illumination from the illumination source, where such affected illumination is detectable by the photodetector array.

In an example, a biomarker detector is provided comprising: a sealable housing; an inlet configured to receive fluid; and a sensor device within the sealable housing and communicatively coupled to the inlet to receive the fluid, the sensor further comprising an illumination source, a photodetector array, and a microfluidic chip positioned between the illumination source and the photodetector array, the microfluidic chip comprising a microfluidic chamber holding a sample/colloidal nanoparticle biosensor mixture solution to permit colorimetric analysis of analyte-induced nanoparticle aggregation while it is aligned with the illumination source and the photodetector array in the biomarker detector architecture.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is an exploded view of a multimodal bioassay sensor device in the form of a smart pipette, in accordance with an example.

FIGS. 2A-2D illustrate exploded views of a multimodal bioassay (bio)sensor device in the form of a smart pipette device in different positions, including a push position (FIG. 2A), a sample collection position (FIG. 2B), a detection position (FIG. 2C), and a tube replacement position (FIG. 2D), in accordance with an example.

FIG. 3 illustrates an example multimodal (bio)sensor that may be used in a smart pipette or other device and that has a microfluidic chip formed of nanoplasmonic barcode detectors and using a plasma separation chamber, in accordance with another example.

FIG. 4 illustrates an example multimodal sensor having a microfluidic chip formed of nanoplasmonic barcode detectors without using a plasma separation chamber, in accordance with another example.

FIG. 5A illustrates an example microfluidic chip as may be used in a multimodal bioassay sensor, in accordance with another example. FIG. 5B illustrates nanoplasmonic gold nano-hemisphere arrays of the microfluidic chip, in accordance with an example. FIG. 5C illustrates absorptions peaks for 4 different biomarker channels of the microfluidic chip, in accordance with an example.

FIGS. 6A-E illustrate operation of an multimodal integrated sepsis bio-optoelectronic (iSBO) assay device, with a sensor integrating a biotunable nanoplasmonic filter and a few-layer MoS₂ photodetector device, in accordance with an example. FIG. 6A is a cross-sectional view of an example iSBO sensor. FIG. 6B is an optical image of an example fabricated sensor. FIG. 6C illustrates principle operation of the sensor of FIG. 6A during an OFF state (left side) and an ON state (right side). FIG. 6D is a plot of a photocurrent variation. FIG. 6E is a standard curve of photo-electronic cytokine immune-sensor incorporating biotunable nanoplasmonic optical filter on a few-layer MoS₂ photodetector.

FIG. 7 illustrates a biomarker analysis system formed of a smart pipette communicatively coupled to a mobile platform through a wireless network, in accordance with an example.

FIGS. 8A-8E illustrate high-sensitivity on-chip biomarker sensor capable of colorimetric measurements, in accordance with an example. FIG. 8A is a schematic of an integrated and miniaturized bio-optoelectronic 4×4 biomarker sensor having array of enzyme chamber and atomic MoS₂ photoconductor. FIG. 8B illustrates an packaged high-sensitive colorimetric sensor array compared to a five-cent coin. FIG. 8C illustrates a standard calibration curve of D-lactate using the integrated high-sensitive colorimetric sensor array and conventional colorimetric detection. FIG. 8D is a plot showing a comparison of calibration curves of D-lactate spiked in PBS, plasma, and serum of swine and humans from healthy donors. FIG. 8E is a plot of clinical sepsis sample vs. D-lactate level measured by the high-sensitive colorimetric sensor using a smart device configuration.

FIG. 9 illustrate IoT-based smart precision medicine system enabled by a point of care biosensor device such as those in FIGS. 1-8, 12, and 14-18, in accordance with an example.

FIG. 10 illustrates a portable biomarker analysis module smart pipette for nanoparticle aggregation-based colorimetric operation, in accordance with an example. The smart pipette wirelessly transmits measurement data to the smartphone and is comprised of a sampling/detecting tube, a biosensor module, a wireless data transceiver, and a plunger button. The architecture of the biosensor module includes a light emitting diode (LED) illumination source, biosensor chip-tip, and MoS₂ photodetector. The miniaturized sensing part enables the detection of biomarkers in a small sample volume with high sensitivity.

FIG. 11 illustrates performance metrics for various state-of-the-art point of care systems in comparison against an example smart pipette in accordance with an example herein (WB: Whole blood, S: Serum, PBS: Phosphate-Buffered Saline, ELISA: enzyme-linked immunosorbent assay).

FIGS. 12A-12G illustrate an integrated ultra-sensitive MoS₂ cancer biomarker detection by near infrared (NIR) plasmonic-optoelectronic biosensor, in accordance with an example. FIG. 12A illustrates a MoS₂ channel photoconduction change due to an increase in NIR optical absorbance accompanying antibody-conjugated plasmonic AuNP aggregation driven by the presence of biomarkers. FIG. 12B illustrates a cross-sectional view of an ultralow-noise, NIR absorbing few-layer MoS₂ photoconductive channel structure. FIG. 12C illustrates optical micrographs of a 14 nm thick, 1 pm long MoS₂ channel with electrodes. FIG. 12D illustrates the spectrum at CCEA=0 (black curve) and 10 ng/mL (red curve). FIG. 12E illustrates the photocurrent at CCEA=0 (black line) and 10 ng/mL (red line). FIG. 12F illustrates a constructed calibration curve based on ΔIph/Iph0 as a function of CCEA. FIG. 12G is a comparison of sample preparation-free, wash-free CEA detection performance between PBS and Whole blood, both spiked by the same analyte concentrations.

FIGS. 13A-13D illustrate a preliminary result of the noise analysis for an example MoS₂ photodetector, as that of FIG. 12A. FIG. 13A illustrates RMS noise values measured for a 3 nm thick MoS₂ photodetector and a 14 nm thick device, as well as that measured for the 14 nm one after O2 plasma treatment. FIG. 13B illustrates noise power density spectra of the 14 nm thick device and a CdS photodetector. FIG. 13C illustrates external quantum efficiency (EQE) spectra of a plasma-doped MoS₂ photodetector in comparison with an undoped control device. FIG. 13D illustrates the system integrated onto a wireless communication circuit board.

FIGS. 14A-14E illustrate NIR sensitive plasmonic nanoprobe for colorimetric cancer biomarker detection, in accordance with an example. FIG. 14A illustrates a schematic of the NIR sensitive plasmonic nanoprobe. FIG. 14B illustrates a scanning electron microscope (SEM) images of SiO₂ particles and NIR sensitive plasmonic nanoprobes, and large-scale production (scale bar=50 nm). FIG. 14C illustrates NIR sensitive plasmonic nanoprobe aggregation driven by antigen biomarker-antibody binding events. FIG. 14D illustrates electric (E)-field distribution near the plasmonic nanoprobe surface. FIG. 14E illustrates an E-field intensity spectrum around an AuNS, a NIR sensitive nanoprobe, and aggregated nanoprobe particles.

FIG. 15A illustrates a fabrication method of a biosensor, and FIG. 15B illustrates a band diagram of a photodetector with pGr/pM/nM/nGr heterostructure.

FIG. 16A illustrates an exploded fabricated layer structure of a biosensor, and FIG. 16B illustrates a band diagram of a photodetector with pGr/pW/nM/nGr heterostructure. Here, WSe₂ and MoS₂ form a type-II heterojunction at their interface. FIG. 16C illustrates an exploded fabricated layer structure of a biosensor like that of FIG. 16A but having an dielectric and gate structure, and FIG. 16D illustrates a band diagram of a gated 2D heterostructure.

FIG. 17 illustrates an exploded fabricated layer structure of another example biosensor, in this instance having a vertically aligned NIR-Transition metal dichalcogenide monolayers (TMDC) photodetector heterostructure.

FIG. 18A illustrates an integrated system biosensor chip in exploded form and having a biomarker detector in the form of a handheld module assembled by inserting layers of a LED chip, a microfluidic biochip with a plasma isolation filter and a sample detection chamber, and a NIR-Transition metal dichalcogenide monolayers (TMDC) photodetector into a cartridge-spacer body frame, in accordance with an example. FIG. 18B illustrates a NIR colorimetry detection principle for the biosensor of FIG. 18A. FIG. 18C illustrates detection steps in an example, application of the biosensor chip of FIG. 18A, in accordance with an example.

FIG. 19A illustrates a smart pipette, in accordance with an example. FIG. 19B illustrates a micro-optics module (μOM) mimicking the integrated system biosensor system of FIG. 18A for software testing. The uOM module incorporates off-the-shelf commercial photodetector (with limited sensitivity) and LED components. FIG. 19C illustrates an app software for smartphone-based data access.

FIGS. 20A & 20B illustrate a smart pipette biosensing module, in accordance with an example. FIG. 20A illustrates geometric optics simulation results showing that micro-lens like biochip flow-cell structure leading to signal amplification. FIG. 20B illustrates a calculated sensitivity indicating that the micro-lens like biochip structure would enable 20 times more sensitive detection than a conventional slab channel structure.

FIG. 21 illustrates a portable biomarker analysis module. FIG. 21 i) illustrates mixing nanoprobes and whole blood in a sample tube. FIG. 21 ii) illustrates inserting the biochip tip. FIG. 21 iii) illustrates pushing the pipette plunger. FIG. 21 iv) illustrates releasing the plunger to load the sample. FIG. 21 v) illustrates incubating the sample and detecting the target biomarker. FIG. 21 vi) illustrates transmitting the data to a smartphone. FIG. 21 vii) illustrates replacing the used biochip with new one.

### DETAILED DESCRIPTION

The present techniques provide blood biomarker analysis systems for fast biomarker identification. In some examples, the blood biomarker analysis system includes a multimodal bioassay device capable of operation in the field, at the point-of-care. The bioassay device may be implemented as a smartphone-connected, highly portable, pipette-shaped platform device, having a multimodal sensor arrangement. The bioassay device may interface with connected computing devices that generate and display medical reports, such as biomarker summary reports based on the bioassay device, for use by medical professionals during patient treatment. The biomarker analysis system may be trained using machine learning algorithms to detect one or more biomarkers and patterns in a very short time window (e.g., within 10 mins, 5 mins, or lower), with high accuracy, to provide further benefit for emergency and chronic care applications. Thus, in some examples, the present techniques provide a biomarker analysis system can be used at the point-of-care, soon after the time of illness or injury and at the location of the illness or injury, to allow medical personnel more accurate assessments of a subject's condition and treatment options.

In some examples, the biomarker analysis system may be used at the point-of-care in the intensive care unit (ICU), an ambulance, and a remote area under limited resources to detect the early onset and predict outcomes of acute illnesses, and situations where sending blood to a lab for analysis is impractical and may cost lives.

In various examples herein, multimodal bioassay devices are able to sensor for one or more biomarkers associated with a subject's injury, trauma, or other condition. Example biomarkers include cytokines (IL-2, INF-γ, IL-4, IL-10, IL-1β, TNF-α, IL-6), complement system biomarkers (C3a, C5a, C5b-9, Bb, C4d) sepsis biomarkers (CRP PCT, CitH3), lung-injury biomarkers (SPD, sRAGE, KL-6, CC-16, PAI-1, protein C, vWF, HMGB1), brain-injury biomarkers (NSE, S100(3, GFAP, UCHL1, BDNF, NFL), metabolites (D-lactate, histamine), and so forth. Example subject conditions associated with and thus identifiable from these biomarkers include, trauma or surgery-induced injury, sepsis/sepsis shock, asthma, systemic inflammatory response disorder (SIRS), acute respiratory distress syndrome (ARDS), cytokine release syndrome (CRS), acute allergic response, and others.

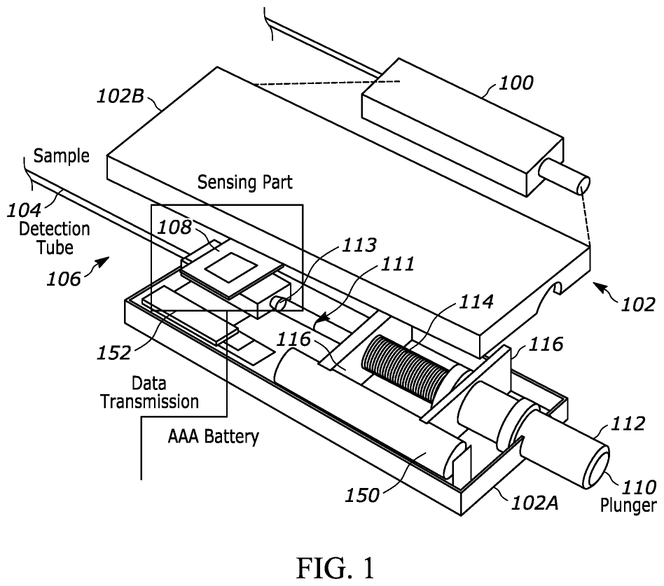

FIG. 1 illustrates an example multimodal bioassay device in the form a smart pipette 100. The pipette 100 is formed of a releasable, sealable housing 102, which, in the illustrated example, has a housing portion 102A and a capping portion 102B that come together to form a sealed fitting during deployment. That is, in some examples, the housing 102 is sealed from fluid contamination of any sort during deployment. In some examples, a hermitically sealing engagement is achieved between the housing portion 102A and the capping portion 102B.

To facilitate blood draw, the pipette 100 includes a detection tube 104 extending from a sensor end 106 of the pipette 100. The detection tube 104 may be held in place and in a sealed engagement by the housing 102. The detection tube 104 may be formed of plastic (PTFE) and is fixedly connected to a sensor 108 at a proximal end and may include an adaptor, Luer connector, or other engagement it a distal end (not shown) for attaching to a blood drawing system.

In the illustrated example, the sensor 108 is fixedly mounted in the housing portion 102A, for example, against an engagement bottom surface thereof. Opposite the fluid accepting connection to the detection tube, the sensor 108 is coupled to a plunger apparatus 110 adjustable between an initial position and a fluid drawing position. The plunger apparatus 110, for example, may be formed with a push button 112 and spring assembly 114, where pushing the push button 112 toward the housing, activates the spring assembly 114 to draw fluid (e.g., blood) through detection tube 104 into the sensor 108 through suction pressure differential. The housing portions 102A and 102B may be configured to form a sealed engagement around the push button 112, for example, through a sealing rubber ring or other friction-based engagement between the push button 112 and circular receiving opening defined by the housing portions 102A and 102B. In other examples, sealing of an inner chamber of the housing may be achieved by one or more sealing structures 116.

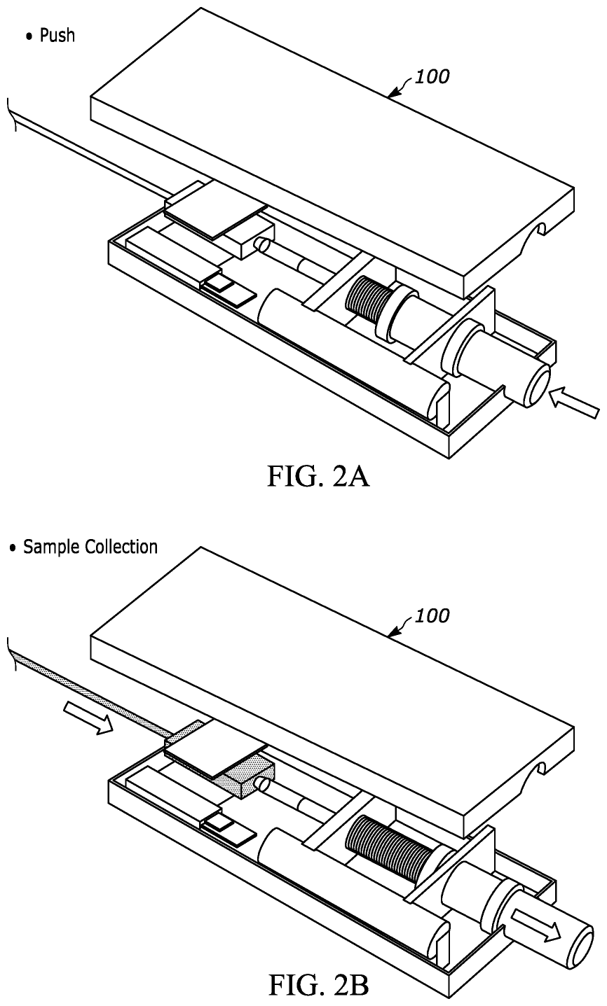

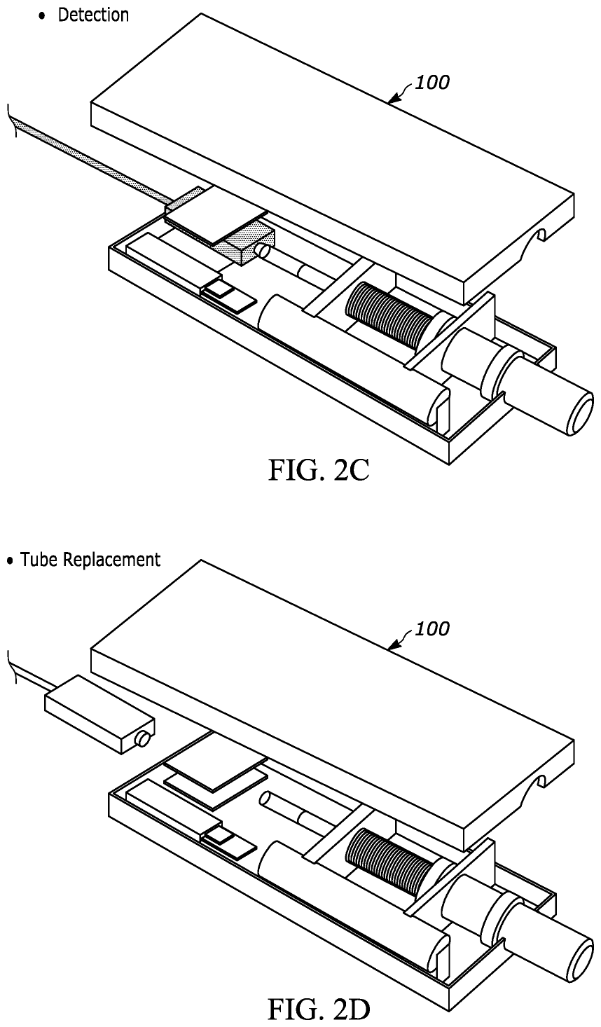

FIGS. 2A-2D illustrate the pipette 100 in four different positions, with the capping portion 102B removed for visualization purposes only. A fluid drawing position is shown in FIG. 2A, where the push button 112 is depressed inwardly toward the housing 102. Pushing the button 112 generates a negative pressure and the fluid is collected (drawn) into the detection tube 104 and into the sensor 108 (as shown) by releasing the push button 112 which causes the depressed push button 112 to extend outwardly from the housing 102 (FIG. 2B). As the fluid enters the sensor 108, the pipette 100 enters a detection state (FIG. 2C), in which the sensor 108 performs bioassay operations to detect for one or more biomarkers in the fluid.

In some examples, the pipette 100 may be a one-time use device, in which the device is discarded after completion of biomarker detection. In some examples, the pipette 100 may be reusable, for example, where the sensor 108 may be removed along with the detection tube 104 and replaced with a replacement sensor and detection tube (FIG. 2D). In some examples, the plunger apparatus 110 includes a receiver end 111 for receiving an outlet tubing 113 integrally formed with the sensor 108, where the outlet tubing 113 is configured to prevent fluid from entering the receiver end 111.

In addition to the sensor, the pipette 100 includes a battery power source 150 and a data transmitter 152 (shown in FIG. 1).

The data transmitter 152 may include one or more processors and one or more memories and may be configured, through software, hardware, or some combination thereof, as a wireless transmitter capable of wireless communications according to any suitable communication protocol, including, by way of example, the many variants of IEEE 802.11 (Wi-Fi), MU-MIMO, Wireless ax, Wireless ad, Message Queue Telemetry Transport (MQTT), ZigBEE, ZWave, Thread, Near Field Communication (NFC), Bluetooth (BT), and Bluetooth Low Energy (BLE).

There are numerous conventional sensor technologies in the art, including nanoplasmonic point of care (POC) biosensor technologies, each with numerous shortcomings. Label-free biosensors, for example, have seen growing interest. With the elimination of labeling agents, such as isotopes, fluorophores, and enzymes, label-free biosensors can avoid adverse effects on biomolecular binding events and error due to the inconsistent binding behavior of labels to analytes, which all theoretically saves money and time. Localized surface plasmon resonance (LSPR) nanoplasmonic biosensors, for example, perform label-free biomarker analysis using various types of sensors, including mechanical (microcantilever, acoustic wave, and quartz crystal microbalance mass), electrical (electrochemical impedance spectroscopy, amperometric detection, capacitive affinity detection, nanoelectronic field-effect transistors), optical (photonic crystal, optical resonator), and plasmonic (surface plasmon resonance, localized surface plasmon resonance (LSPR)) sensors. Compared to other label-free sensors, LSPR-based nanoplasmonic biosensors have been shown to be particularly advantageous for POC measurements. They are robust, rapid, cost effective, easy to integrate into miniaturized fluidic devices with simple optics, and well suited for multiplex biomarker measurements. These biosensors are attractive for diagnosing and trajectory monitoring of critically ill patients as they allow for a biomarker measurement within 20-40 minutes. An example implementation of LSPR sensors is described in U.S. Application Ser. No. 62/489,872, entitled, “Systems and Methods For Performing Immunoassays,” and in PCT/US2018/028856, filed Apr. 23, 2018, the entirety of both of which are hereby incorporated by reference. In contrast, non-label-free techniques represented by enzyme-linked immusorbent assay (ELISA) normally require a long assay time and many steps in a centralized clinical laboratory setting. To make the assay time as short as that of label-free biosensing in a miniaturized device (e.g., microfluidic) setting, some research groups have introduced sandwich immunoassay protocols with one-step mixing of all reagents. However, these protocols resulted in 10-1,000-fold reduction of the detection sensitivity as compared to the widely accepted regular ELISA protocol.

Other conventional systems include nanoplasmonic POC immune biosensors. A truly portable and self-containing system should incorporate several key components, including LSPR bionsensing nanostructures, a fluidic system for sample handling, optical components, a light source, a photodetector for signal reading, and a photo-signal processing electronic unit. Table 1 summarizes state-of-the-art LSPR-based POC immunosensor systems found in the literature. The most common detector used for these systems is a smartphone. Because smartphones contain a built-in LED flash light source, a CMOS (complementary metal oxide semiconductor) imager, and embedded central processing and graphics processing units, they facilitate rapid, user-friendly analyte detection and wireless data transmission. At present, >2 billion people possess a smartphone, making smartphone-based signal detection and processing the most rational approach because it would enable the most people, even those in developing countries with limited resources, to access POC systems.

There are many recognized shortcomings of current technology. For example, although the existing POC immune biosensor systems show promise to some degree (some with an impressively short assay time), their implementations in real clinical settings are yet to be realized. Previous work on LSPR-based POC testing largely focused on the development of “proof-of-concept” laboratory prototype devices. None of them have been used for multiplex analysis of sepsis-relevant blood biomarkers in a POC setting. Furthermore, the optical signal detection using a phone camera suffers a relatively low signal-to-noise ratio resulting from a high level of background noise in CMOS image sensors (unlike the photoconductive nanosheet channel-based detection proposed here). Hence, the existing state-of-the-art, portable LSPR POC devices unquestionably have much lower sensitivity than the gold standard ELISA. Biomarker detection for rapidly evolving and dynamic disease states like sepsis must meet more stringent requirements compared to cancer and other chronic disease diagnoses. Specifically, the limit of detection must be at least comparable to that of the gold standard Enzyme-linked immunosorbent assay (ELISA) assays with a much faster sampling-to-answer time of <30 minutes (excluding the serum sample preparation time of ˜30 min). Rapidly detecting biomarkers in highly dynamic diseases such as sepsis within these specifications is not feasible with existing technologies and prototypes. In various examples herein, however, the sensor configurations of smart pipettes are able to overcome these deficiencies in the art.

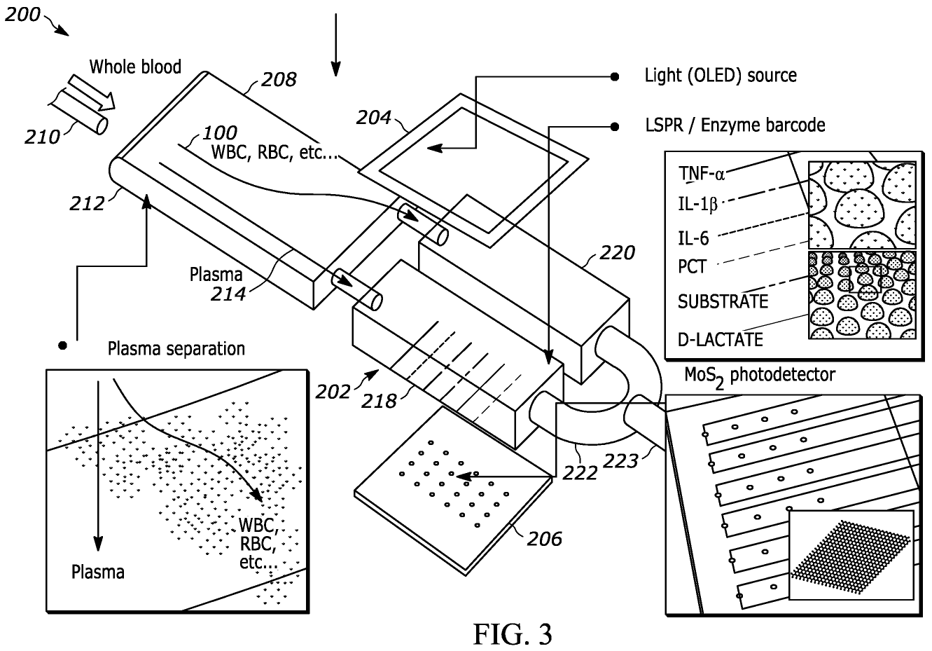

FIG. 3 illustrates an example multimodal sensor 200 that may be used in place of the sensor 108 of FIG. 1. In an example, the sensor 200 includes a microfluidic chip 202 disposed between an illumination source 204, and a photodetector array 206. In the illustrated example, the illumination source 204 is a light emitting diode (LED) source in the form of an electronically controllable polychromatic illumination source capable of generating an illumination over a range of wavelengths such as a visible region of about 380 nm to about 750 nm, near infrared from about 750 nm to about 1.4 nm, specifically, 450 nm, 532 nm, 600 nm, and 650 nm. In the example of FIG. 3, the illumination source 204 is an organic light emitting diode (OLED) source.

In the example of FIG. 3, the microfluidic chip 202 is a nanoplasmonic barcode chip having a plurality of different nanoplasmonic barcode detectors, each configured through antibody conjugation to capture a different biomarker. In the illustrated example, six antibody barcode detectors are shown (see inset) each formed as a linear barcode-shaped LSPR biosensor pattern, formed in rows parallel to one another, and generally orthogonal to a main direction of fluid flow. In an example, the microfluidic chip 202 may include antibody barcodes for capturing TNF-α, IL-1β, IL-6, PCT, substrate, and D-Lactate (as shown). Moreover, any number of example biomarkers may be used, including, as referenced above, cytokines (IL-2, INF-γ, IL-4, IL-10, IL-1β, TNF-α, IL-6), complement system biomarkers (C3a, C5a, C5b-9, Bb, C4d) sepsis biomarkers (CRP PCT, CitH3), lung-injury biomarkers (SPD, sRAGE, KL-6, CC-16, PAI-1, protein C, vWF, HMGB1), brain-injury biomarkers (NSE, S100β, GFAP, UCHL1, BDNF, NFL), metabolites (D-lactate, histamine), and so forth. In another example, the microfluidic chip 202 may include antibody barcodes for capturing cancer biomarkers including, by way of example and not limitation, carcinoembryonic antigen (CEA), cancer antigen (CA)19-9, prostate-specific antigen (PSA, Pro2PSA), a human epidermal growth factor receptor (HER)2, AFP, (CA)125, HE4, OVA1 test, ROMA test, (CA)15-2, (CA)27-29, Tg, and hCG. In other examples, viral protein markers may be captured. In yet other examples, the microfluidic chip 202 may include antibodies, monobodies, or other particles (including for example nanoparticles) for capturing various different viral proteins, such as SARS-Cov and SARS-Cov-2 spike protein, MERS-CoV spike protein nucleocapsid protein, HIV-1 protein, gp41, no binding to Tat, TAR RNA, intracellular Nef, p24, minimum binding to Tat, Tat, etc. to list a few. For example, antibodies such as peptide SP1- and SP4-induced polyclonal antibodies have been shown to capture the SARS-Cov and SARS-Cov-2 spike protein, see, e.g., Chang M S, Lu Y T, Ho S T, et al. Antibody detection of SARS-CoV spike and nucleocapsid protein. Biochem Biophys Res Commun. 2004; 314(4):931-936. doi:10.1016/j.bbrc.2003.12.195; Juanjuan Zhao, Quan Yuan, Haiyan Wang, Wei Liu, Xuejiao Liao, Yingying Su, Xin Wang, Jing Yuan, Tingdong Li, Jinxiu Li, Shen Qian, Congming Hong, Fuxiang Wang, Yingxia Liu, Zhaoqin Wang, Qing He, Zhiyong Li, Bin He, Tianying Zhang, Yang Fu, Shengxiang Ge, Lei Liu, Jun Zhang, Ningshao Xia, Zheng Zhang, Antibody Responses to SARS-CoV-2 in Patients With Novel Coronavirus Disease 2019, Clinical Infectious Diseases, ciaa344, https://doi.org/10.1093/cid/ciaa344

The microfluidic chip 202 may be formed of a substrate formed of poly (methyl methacrylate) (PMMA), polycarbonate (PC), polystyrene (PS), polyvinyl chloride (PVC), polyimide (PI), and the family of cyclic olefin polymers (i.e., cyclic olefin copolymer (COC), cyclic olefin polymer (COP), and cyclic block copolymer (CBC)) and Glass with a rectangular-, cylindrical-, or conical-feature.

In the illustrated example, the microfluidic chip 202 is formed of a substrate 208 that is fed by an inlet channel 210 connected to the detection tube for receiving whole blood, or other fluid, during detection. The downstream of the inlet channel is a plasma separation chamber 212 configured to separate plasma from white blood cells (WBC), red blood cells (RBC), and platelets. The chamber 212 feeds a plasma channel 214 and a remaining fluid channel 216. The plasma channel 214 feeds a multimode biomarker detection chamber 218 having multiple barcode-shaped nanoplasmonic biosensor patterns, each for detecting a different biomarker, a substrate pattern, or a D-Lactate enzymatic reagent pattern. The separation chamber 212 allows for avoiding false positive signals resulting from the settling of WBC, RBC, and platelets within the biomarker detection chamber 218. The separated WBC, RBC, etc. are collected in the cell collecting chamber 220 which is placed next to the biomarker detection chamber 218. The end of the biomarker detector chamber 218 and the cell collecting chamber 220 are connected through the curved channel 222, which is connected to a waste at the end of the pipette tip. Negative pressure from the main pipette body goes into the biomarker detector chamber 218 and the cell collecting chamber 220 through the curved channel 222 to generate the fluidic flow manually. The curved channel 222 may be connected to a plunger apparatus (not shown), such as the plunger 110 through an outlet tubing 223 connected to a receiver end of the plunger apparatus.

In an example, the photodetector array 206 is MoS₂ photodetector channel array. A MoS₂ photodetector channel array can be formed as a monolayer structure, where a MoS₂ monolayer operates as a direct-bandgap semiconductor due to quantum-mechanical confinement. Furthermore, as a monolayer, the direct bandgap structure allows for a high absorption coefficient and efficient electron-hole pair generation under photoexcitation. Furthermore, a MoS₂ photodetector channel array can be implemented with a photo-responsiveness across a range of frequencies, including from about 400 nm-about 680 nm, thus allowing for multimodal operation and more accurate operation, in general.

The photodetector array 206 may be configured as a channel array having a pattern that places rows of photodetector elements (arrays) aligned with each of the nanoplasmonic barcode detectors for separate detection of illumination of each of the nanoplasmonic barcodes detectors. In an example, the MoS₂ photodetector channel array was formed of an ultrasensitive MoS₂ nanosheet photodetector channel arrays on a silicon substrate using a nanofabrication technique, whose signal acquisitions, signal analyses, and data transmissions are achievable by a wirelessly connected smartphone or other portable device. A smartphone application may enable user-friendly, nonresource-demanding quantification of the blood biomarkers.

In examples, the proposed photodetector arrays have high uniformity in their photo-response parameters, such as short-circuit photocurrent (Isc), open-circuit voltage (Voc), and responsivity. The relative detector-to-detector variation of these parameters is <10%, <5%, or <1% over the whole chip.

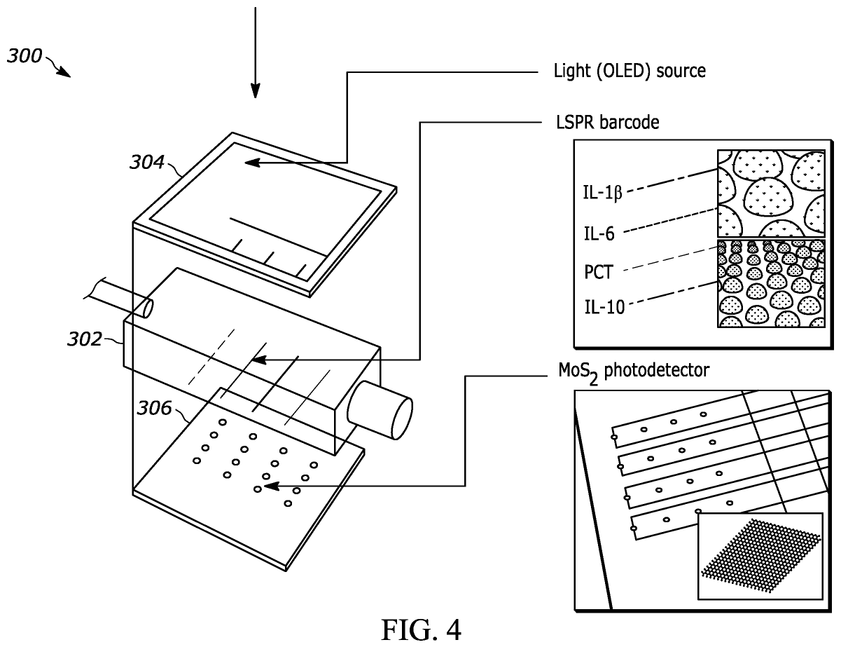

FIG. 4 illustrates another example multimodal sensor 300 similar to the sensor 200 and that represents the sensor 108 of FIG. 1 and has a microfluidic chip 302 disposed between an illumination source 304, and a photodetector array 306. The illumination source 304 and the array 306 may be like the similar elements in FIG. 3. The microfluidic chip 302 is similar to the chip 202, except instead of a plasma separation chamber, during detection, the whole blood enters a nanoplasmonic barcode detector stage 308 having multiple channels, each for detecting a different biomarker, substrate, or D-Lactate, etc.

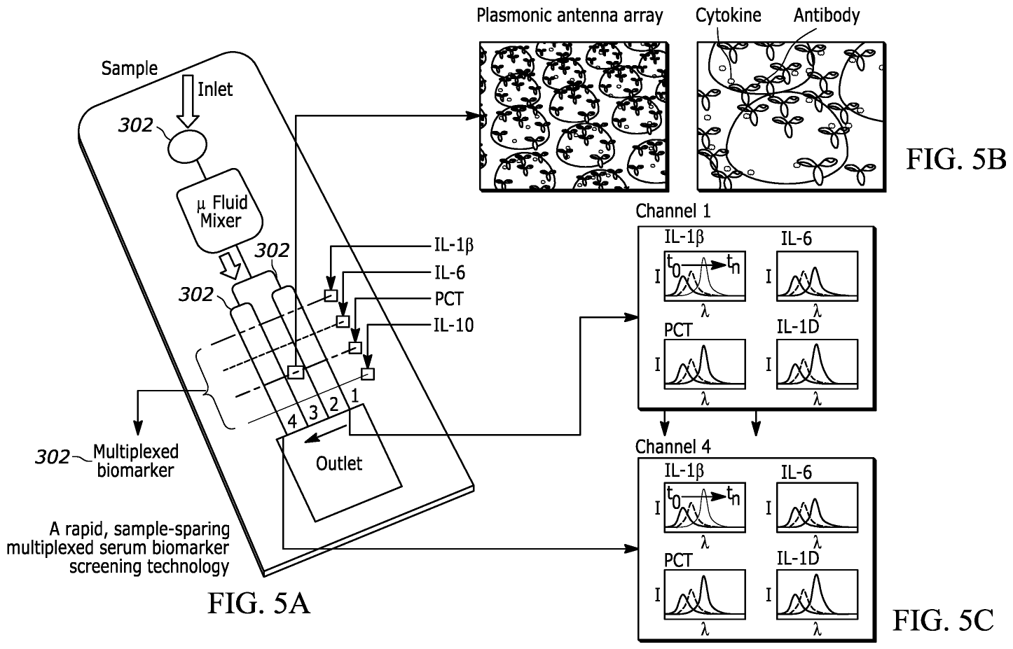

FIG. 5A illustrates an example microfluidic chip 400 configured as a multimodal integrated sepsis bio-optoelectronic (iSBO) assay. In the illustrated example, the chip 400 is a nanoplasmonic biosensor microfluidic chip fabricated using polydimethylsiloxane-based soft lithography and plasma surface treatment-based polydimethylsiloxane-glass bonding. The chip 400 includes an inlet 402, detection channels 404 in a branch splitting (or multiplexing) configuration into four channels, and barcode-shaped patterns 406 of structurally engineered nanoplasmonic gold nano-hemisphere arrays on a glass substrate (FIG. 5B) spanning each of the detection channels 404, each conjugated with a different capture antibody and a reservoir. A scalable gold nanostructural self-assembly technique may be used to engineer the arrayed gold nano hemisphere structures on a substrate. In an example, the dimension (width×height) of the channel is designed to be 500×50 μm². In operation, a small volume (<204) of serum sample (e.g., whole blood) is loaded to each device inlet channel separating into four branch fluidic channels to permit quadruple measurements. The multiplex barcode-shaped biosensor patterns forming the multiplexed biosensor 406 may be orthogonally placed on the bottom of branch channels. To construct such a device structure, plasmonic gold nano hemisphere array structures may be patterned covering a large area of the glass substrate into multiple barcode shapes (for example, four barcode shapes) using a stencil mechanical mask. In the illustrated example, the chip 400 has four LSPR barcodes conjugated with antibodies to target IL-1β, PCT, IL-6, and IL-10 (i.e., representative sepsis biomarkers) co-existing in blood serum. This will form 16 (4 channels×4 barcodes) of nanoplasmonic biosensing pixels. As target biomarker molecules in the blood serum bind to specific sensor capture antibodies, a change of local refractive index near the surface of each biosensing spot will yield an LSPR spectral peak shift. This peak shift will lead to an absorption change of incident light from a light source. Thus, the biosensing pixels will function as optical filter arrays whose transmission characteristics are tuned by the surface binding of their specific target biomarkers. Example absorption peaks for each of the 4 channels for each of the 4 barcodes is shown in FIG. 5C.

Any of the sensors with multimodal bioassay chip like that of FIGS. 3-5 can be implemented in a pipette configuration like that of pipette 100 in FIG. 1.

Sensors with a multimodal bioassay chip like that of FIGS. 3-5 are able to address the critical need for novel detection technology to reduce incidence and severity of sepsis and other acute and chronic inflammatory conditions. These designs are able to provide rapid and time-series detection of blood biomarkers, including for field-deployable biomarker-guided sepsis-inflammatory condition prediction outside a hospital setting to continue in hospital. Such sensors enable early detection of sepsis or other worsening inflammatory condition, allowing early treatment with guided endpoint resolution to reduce the risk of septic shock and multiple organ dysfunction syndrome, thus improving outcomes in acute illness and injury. Furthermore, the sensors are able to provide multi-biomarker testing that can greatly assist in reevaluating opportunities for developing new therapeutics such as blocking agents (etanercept [TNF-α], anakinra [IL-1r] and tocilizumab [IL-6]) and other approaches. Further, unique integration of new markers such as D-Lactate (a bacterial metabolic byproduct) with biomarkers can provide critical context to changes in inflammatory markers given its association with intestinal bacterial translocation and the importance of the gut microbiome in sepsis and other injury conditions, and may be used for therapeutic modulation. Further still, the strategic integration of time-series biomarkers of inflammation coupled with traditional markers and physiology allow precision phenotyping and the development of new precision therapies for sepsis and other complex states of inflammation and immune dysfunction.

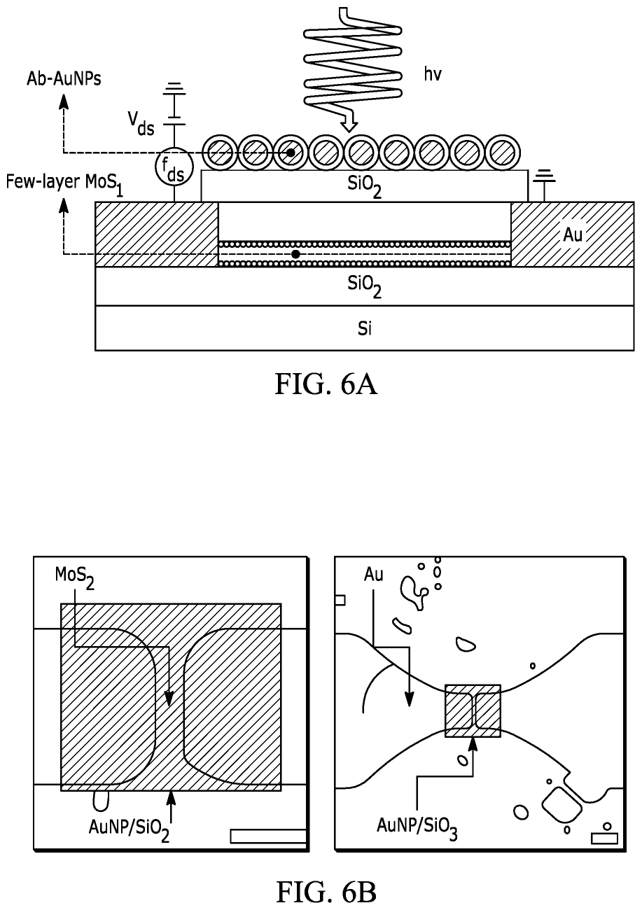

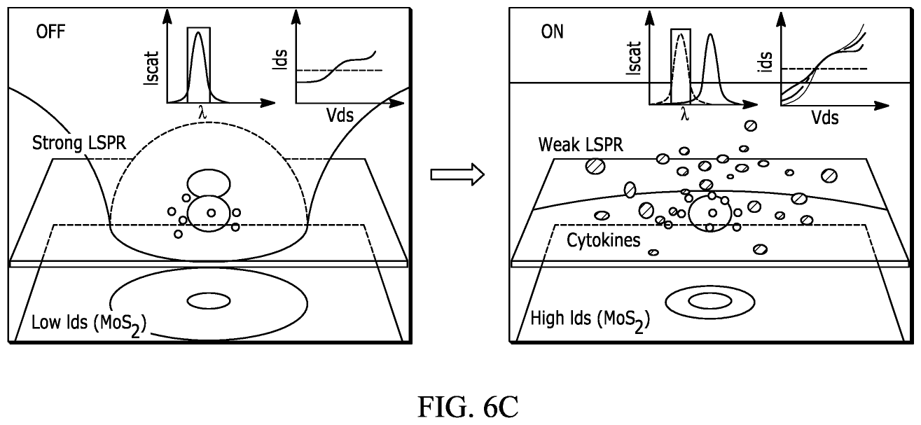

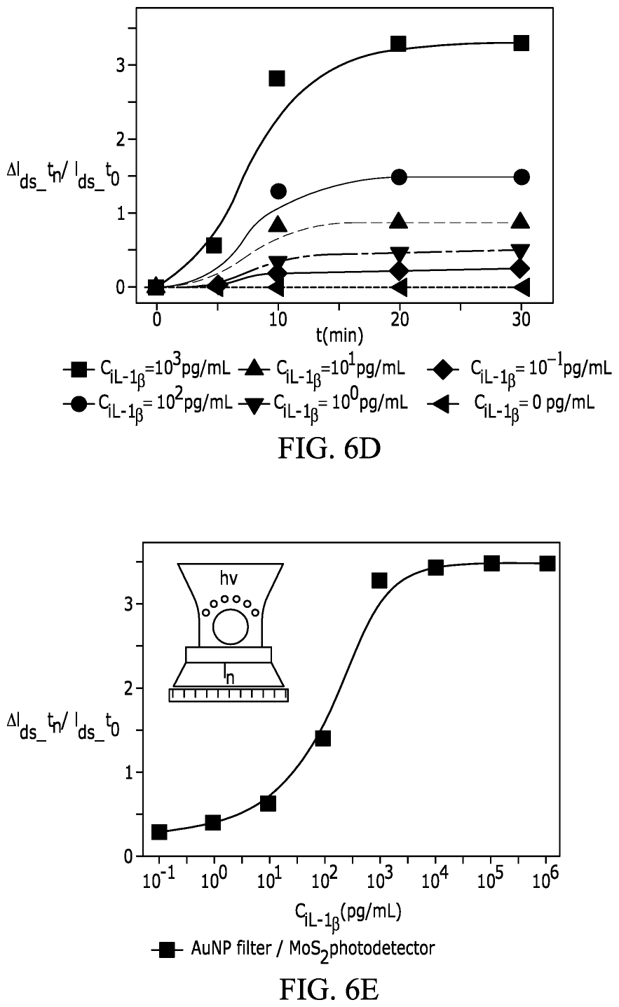

FIGS. 6A-E illustrate operation of an example iSBO multimodal assay device with a sensor integrating a biotunable nanoplasmonic filter and few-layer MoS₂ photodetector device. In the illustrated example, IL-1β detection in biotunable nanoplasmonic optical filter on few-layer MoS₂ photodetector is shown. FIG. 6A is a cross-sectional view of an example iSBO sensor that can be used within a smart pipette. For example, plasmonic gold nanoparticles (AuNPs) may be configured in a linear barcode detection disposed above a few layer MoS₂ photodetector layer responsive to photon incident from above the AuNPs. FIG. 6B is an optical image of an example fabricated sensor, showing a bar size=20 μm. FIG. 6C illustrates principle operation of the sensor of FIG. 6A during an OFF state (left side) and an ON state (right side). Insets show plots of Ids vs. Vds curves of the few-layer MoS₂ photodetector at different IL-1β surface binding incubation time points for a fixed IL-1β concentration of C_{IL-1β}=10 μg/mL. FIG. 6D is a plot of a photocurrent variation (ΔIds_t/Ids_0) during IL-1β surface binding incubation for different CIL-1β values. FIG. 6E is a standard curve of photo-electronic cytokine immune-sensor incorporating biotunable nanoplasmonic optical filter on a few-layer MoS₂ photodetector.

Major advances in the ability to detect tumor-derived biomarkers in the circulation has driven the development of minimally invasive cancer diagnostic methods called “liquid biopsies”. Sensitive assays capable of detecting rare cancer specific analytes immersed in many analytes derived from normal cells are the key to these advances. The analytes used for liquid biopsy include circulating tumor cells (CTCs), cell-free tumor DNA (ctDNA), proteins, metabolites, exosomes, mRNA, and miRNAs. Many conventional liquid biopsies detect ctDNA indicating genetic alterations because of the ease with which DNA molecules are isolated in comparison to other analytes. However, a major fraction of early-stage tumors does not release detectable amounts of ctDNA, even when extremely sensitive techniques are used to identify them. This has kept liquid biopsy from being readily available for discovering cancers at an early stage. In contrast, the literature shows that many protein biomarkers are potentially useful for early detection and diagnosis of cancer in the literature. For example, carcinoembryonic antigen (CEA), cancer antigen (CA)19-9, prostate-specific antigen (PSA, Pro2PSA), a human epidermal growth factor receptor (HER)2 have been extensively studied as analytes to assess cancers through blood tests. Other cancer biomarkers include AFP, (CA)125, HE4, OVA1 test, ROMA test, (CA)15-2, (CA)27-29, Tg, and hCG. Additionally, these biomarkers have been approved for assessing tumor burden in patients already diagnosed with cancer, particularly during therapy in patients with advanced cancer. Carefully selecting a panel of several protein biomarkers, a recent study has proved that blood test is particularly useful for discriminating cancer patients from healthy controls. This multi-analyte blood test has been shown to yield sensitivity nearly 98% to ovary and liver cancers, and 70% to stomach, pancreas, and esophagus cancers with data obtained from more than 1,000 patients. Advances in mass spectrometry are expected to make a new generation of protein biomarkers for cancer available soon. Multiplexed POCT (i.e., POC test capable of simultaneously detecting multiple analytes) can be used to target the above-mentioned proteins (CEA, CA19-9, PSA, and HER2) for cancer diagnosis.

The research community has seen a growing interest in label-free biomolecular analysis techniques. This interest has emerged along with a wider awareness of technical and practical advantages offered by label-free biosensing. With the elimination of labeling agents, such as isotopes, fluorophores, and enzymes, label-free biosensors can avoid adverse effects on biomolecular binding events and error due to the inconsistent binding behavior of labels to analytes, thus saving money and time. Label-free biomarker analysis has been performed using various types of sensors, including mechanical (microcantilever, acoustic wave, quartz crystal microbalance mass), electrical (electrochemical impedance spectroscopy, amperometric detection, capacitive affinity detection, nanoelectronic field-effect transistors), optical (photonic crystal, optical resonator), and plasmonic (surface plasmon resonance, localized surface plasmon resonance [LSPR]) sensors. Compared to other label-free sensors, LSPR-based nanoplasmonic biosensors are particularly advantageous for POC measurements. In LSPR biosensing, the surface binding of analyte molecules is detected in real time from a shift in photon absorbing and scattering behaviors of collectively oscillating conduction-band electrons highly localized on the surfaces of metallic nanoparticles. These biosensors permit ref ractometric detection of concentration-dependent biomolecular surface binding and sensor miniaturization, both leading to rapid and sample-sparing analyte analysis. They are robust, rapid, and cost effective, making them easy to integrate into miniaturized fluidic devices with simple optics.

Returning to FIG. 6A, a label-free optoelectronic biosensor is provided that combines LSPR biosensing and optoelectronic effects for protein biomarker analysis. The device includes (i) an optically transparent glass layer (SiO₂) on which antibody conjugated gold nanoparticle arrays (AuNPs) are formed and (ii) a two-dimensional (2D) transition metal dichalcogenide (TMDC) photoconductive channel of a few atomic layers underneath the glass layer, as shown for example in FIGS. 6A and 6B. Light transmission variations due to LSPR peak shifts induced by protein surface binding onto nanoparticles on the glass layer were detected by the 2D molybdenum disulfide (MoS₂) channel in the device. 2D layered materials, such as graphene and TMDCs are highly attractive due to their superior electronic, optoelectronic, chemical, mechanical properties as well as their 2D structure that is compatible to state-of-the-art planar micro/nanofabrication techniques. In particular, 2D materials, when serving as sensing channel or electrode materials, exhibit an extremely low level of internal electronic noise. This is attributed to the fact that the surfaces of 2D materials have an extremely low density of dangling bonds. Such a unique 2D surface property is expected to provide the active sensing channels with low densities of scattering centers (and hence low Flicker noise level), and enables highly-sensitive, low-noise-level detection of analytes (i.e., a high signal-to-noise ratio). Citrullinated histone H3 (CitH3), a protein biomarker released to the blood circulatory system by neutrophils (one of white blood cell types) upon bacterial infections, may be detected using the label-free optoelectronic biosensor. The biosensor achieved a limit of detection (LOD) of 0.87 pg/mL (56 fM) for CitH3, which is 250-fold lower than that of commercial enzyme-linked immunosorbent assay (ELISA) (the current gold standard technique for protein detection), a large dynamic range of 105, a short sample-to-answer time of 20 min, and a small sample volume of 2.54.

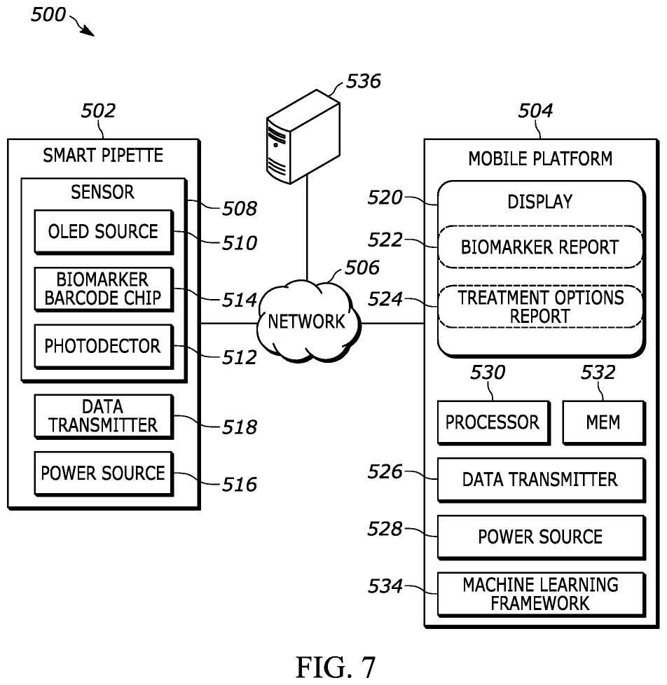

FIG. 7 illustrates biomarker analysis system 500 formed of a smart pipette 502, like that of the smart pipette 100, communicatively coupled to a mobile platform 504 through a wireless network 506. The smart pipette 502 includes a biosensor 508 like that of FIGS. 3-6, which in the illustrated example includes an OLED illumination source 510, a photodetector 512 and a biomarker barcode detector chip 514 that may be formed of one or more nanoplasmonic barcode detectors, in accordance with examples herein. The smart pipette includes power source 516 and data transmitter 518 configured for wireless communication with the network 506. The data transmitter 518 may include one or more processors and one or more memories to control operation of the

The mobile platform 504 may be mobile computing device, including a personal computer, tablet, wearable smart device, smartphone, etc. The mobile platform 504 includes a display 520 that may display various displays and interfaces to a user, including, by way of example, a biomarker report 522 and a treatment options report 524. The mobile platform includes a data transmitter 526 and a power source 528, as well as separate processor(s) 530 and memory 532.

In an example, the biomarker system 500 is configured as a point of care sepsis diagnostic system, that provides a self-contained, portable, multi-biomarker detection device in the form of the smart pipette 502 that upon activation, e.g., depression of a triggering plunger, captures target biomarkers at antibodies on barcode surfaces of a sensor, where under controlled illumination, biomarker binding events leading to changes in the LSPR spectral intensity are recorded by nanosheet photodetector channel arrays, and the measured signal change is transmitted to the mobile platform 504 and processed through calibration curves for each of the biomarkers (e.g., IL-1β, PCT, IL-6, and IL-10). As a smartphone, for example, the mobile platform 504 may display concentration values of the biomarkers in the biomarker report 522.

In some examples, the mobile platform includes a machine learning framework 534 that includes machine learning algorithms for analyzing data collected from the sensor 508, such as including levels of circulating biomarkers in blood, as well as other stored and/or sensed data on the mobile platform 504. In some examples, the machine learning framework 534 is configured to augment diagnostic and prognostic accuracy by providing classifiers based on this data to establish a panel of biomarkers enabling personalized or precision medicine in the diagnosis, trajectory monitoring and treatment of sepsis and other inflammatory/immune based disorders. Conventional machine learning algorithms for prediction-based retrospective tests fail to achieve levels of precision that are actionable. However, using machine learning algorithms and time-series detection using the smart pipette and biosensors herein, an accurate machine learning based sepsis diagnosis and suggested treatment medicine system is now achievable. The machine learning framework, for example, may be taught based on a comparison of time-series smart pipette biomarker panel for multiple patient cohorts. Even with small sample size, performing a univariate analysis using logistic regression, allows for evaluation of each biomarker using area under the receiver operating characteristic curve (AUROC) and area under the precision-recall curve (AUPR) using leave one out cross validation. With such configurations, the single most predictive biomarker or a subset combination of biomarkers that provide prediction may be identified. In some examples, such processes are performed by the machine learning framework 534 during training. Further, by performing regularized multivariate logistic regression using all biomarkers and comparing the AUROC and AUPR using leave one out cross validation we have shown the utility of jointly analyzing all biomarkers, as may be further determined as a part of a training process of the machine learning framework 534. From the analysis of a predictive biomarker or a combination of biomarkers that provided prediction, the machine learning framework 534 may then be established with trained classifiers for accurate diagnosis and prediction of the sepsis trajectory, for example, by incorporating clinical variables using enriched data from the electronic medical record (routine laboratory values, vital signs, comorbidities, demographics, and treatments). Further still, in some examples, a non-linear classification process (e.g., gradient-boosting trees) may be used by the machine learning framework 534 to distinguish between biomarker signatures between the sepsis and non-sepsis (but inflammatory) or any other condition under examination. For example, data can be correlated with sepsis severity, treatments, and outcomes, and this can serve as a basis for future trials in the diagnosis and precision phenotyping of sepsis. The machine learning framework 534, as trained, may classify subjects, from their biomarker data from the smart pipette 502, among sepsis patients as classifications: mortality, high sepsis severity, ICU stay >2 days, or need for advanced therapies such as vasopressors, for example. In some examples, the machine learning framework 534 may perform survival analysis using a regularized person-time logistic regression model. Further still, the machine learning framework 534 may be configured to recommend a listing of possible treatment options in the report 524, based on the biomarker data in report 522. In some examples, the mobile platform 504 may communicate with network accessible server 536 for storing biomarker data, biomarker reports, treatment options reports, etc. In some examples, one or more of the processes described herein may be performed by the server 536 interfacing with one or both of the smart pipette 502 or the mobile platform 504, including the processes of the machine learning framework 534 with may alternatively be implemented in the server 536.

The system 500 provides for analysis and diagnosis of staging of a condition, such as sepsis, while allowing delivery of appropriate therapies at dose amounts suitable to the patient's immune response, where those therapies may differ from subject to subject and where the dose amounts may different from subject to subject, as well as during improvement or degradation of the subject's condition.

In some examples, the present techniques provide a biomarker analysis system forming a smartphone-connected, highly portable point-of-care diagnostic platform device. These techniques strategically integrate nanomaterial-based miniature components to form an optoelectronic biosensor pixel. In an example, one component is an optical glass window layer coated with structurally engineered metallic nanoparticle arrays that exhibits biologically tuned nanoplasmonic light absorbance resonance shifts accompanying 1000-fold near-surface electric field enhancement. Another component is a mechanically printed array of two-dimensional few-layered semiconducting transition metal dichalcogenide (TMDC) nanochannels on a silicon substrate that permits high-responsivity, high-quantum-efficiency photoelectronic conversion at extremely low noise (1000 times lower than reported in the literature). Biomarker analysis system integration may be achieved by contactless optical coupling between the two components, which can prevent unwanted electrical shorting during a wet biological measurement.

The smart pipette devices herein enable label-free, concurrent detection of multiple protein biomarkers at unprecedented levels of detectability (LOD<1 pg/mL˜50 fM) and response speed (<10 min) in point-of-care settings. This sensor response speed is equivalent to a total assay time more than 20-100 times shorter than that of the conventional ELISA gold standard technique.

The techniques herein may be implemented in any number of uses owing to their general applicability to assays involving receptor-analyte interactions. The receptor types used in accordance with the present techniques, for example, can be readily extended to a wide variety of antibodies, peptides, and oligonucleotides. This will allow these techniques to be implemented for other biological assays than cytokine protein biomarker analyses, such as receptor-ligand assays, enzyme assays, and DNA assays. In addition to protein binding assay, we have demonstrated that the system allows high-sensitivity on-chip colorimetric measurements of small metabolite molecules (e.g., D-lactate) in one of its assay modality modes.

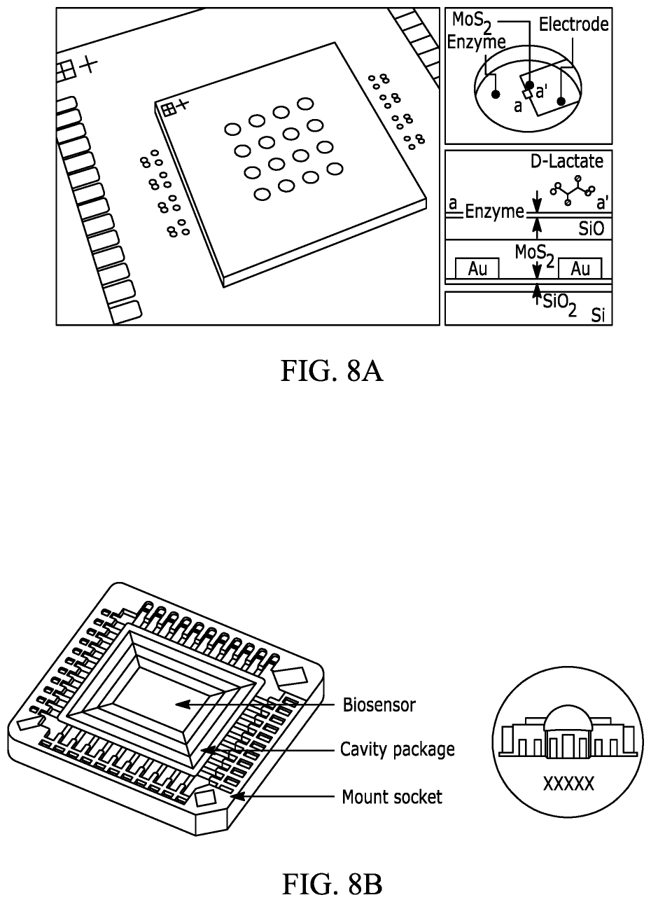

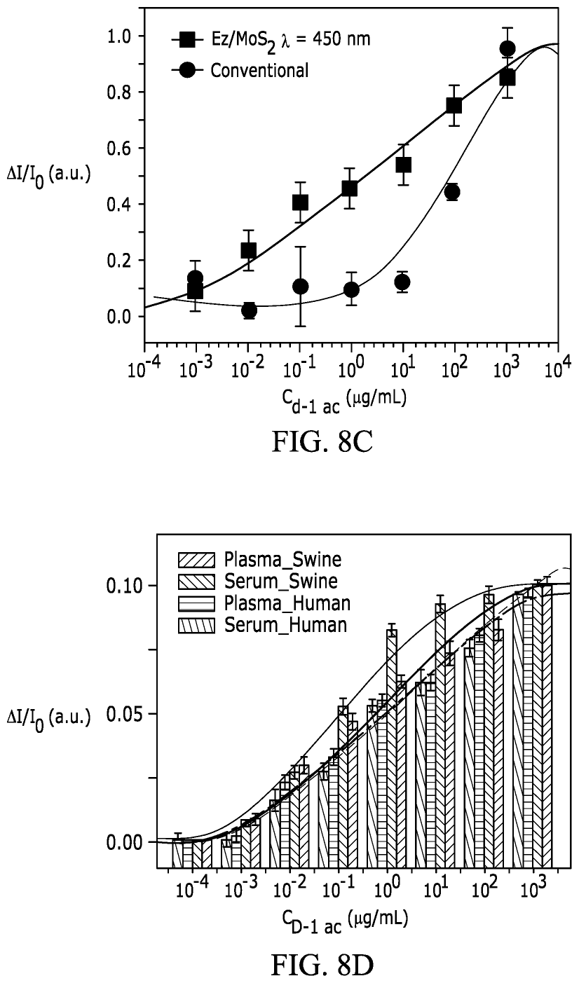

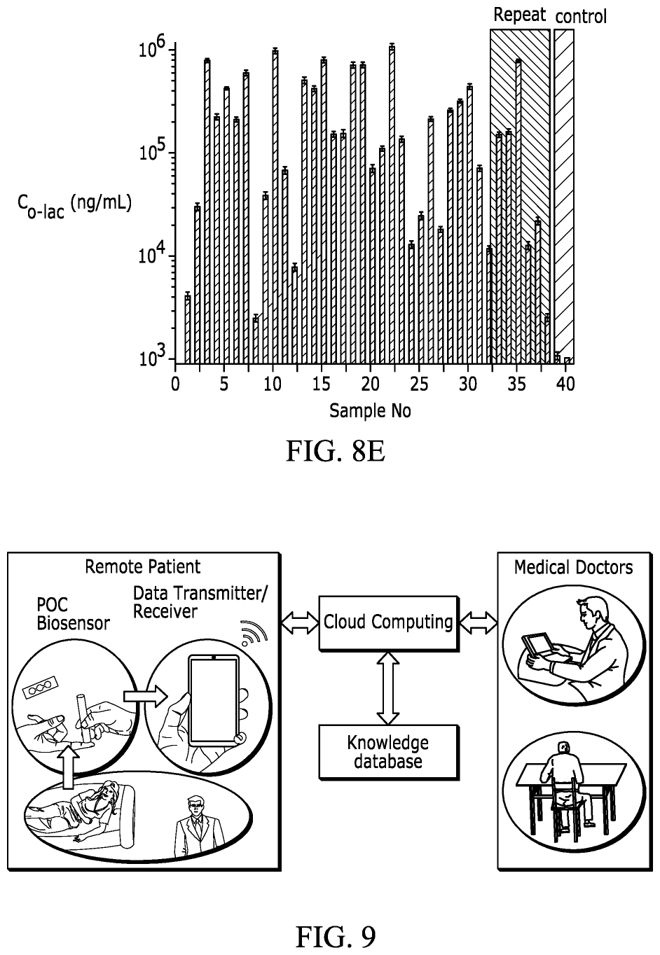

FIGS. 8A-8E, for example, show high-sensitivity on-chip colorimetric measurements, in an example. FIG. 8A is a schematic of an integrated and miniaturized bio-optoelectronic 4×4 array of enzyme chamber and atomic MoS₂ photoconductor (scale bar=2.5 mm). The integrated and miniaturized Ez/MoS₂ sensor array will be integrated into a POC device assembled by a smartphone-based data display system. In FIG. 8B, the image of a packaged high-sensitive colorimetric sensor array in the chip carrier mounted on a socket (3M) (scale bar=1 cm) compared with that of a five-cent coin is shown. FIG. 8C illustrates a standard calibration curve of D-lactate using the integrated high-sensitive colorimetric sensor array and conventional colorimetric detection. FIG. 8D is a plot showing a comparison of calibration curves of D-lactate spiked in PBS, plasma, and serum of swine and humans from healthy donors. FIG. 8E is a plot of clinical sepsis sample vs. D-lactate level measured by the high-sensitive colorimetric sensor using a smart device configuration like that of FIG. 3 or 4.

The biomarker analysis systems herein may be implemented for use at the point of care or near point of care in the intensive care unit (ICU), general ward, emergency department, clinical laboratory, an ambulance, and a remote area under limited resources to detect the early onset and predict outcomes of acute illnesses, such as injury, surgery, sepsis/sepsis shock, asthma, systemic inflammatory response disorder (SIRS), acute respiratory distress syndrome (ARDS), cytokine release syndrome (CRS), and so forth.

The application of the Internet of Things (IoT)-based technologies in medicine promises to advance human healthcare, enabling real-time data acquisition and sharing by means of information networks. The real-time data allow for the monitoring and error-free precision/personalized treatment of patients outside a hospital. This could drastically shift the way of screening and monitoring cancer from a hospital setting-based approach to a personal location-based approach. IoT-based healthcare can monitor human diseases and prevent them at an early stage from advancing to a lethal stage in a “smart city” infrastructure using a network of remotely connected POC or wearable biosensors. For example, FIG. 9 illustrates a scenario where cancer biomarker data of a patient is collected in a home or local clinic (via a computing device 600 at the location) under limited medical resources, transmitted to a remotely located hospital center (for example a medical doctor computing station, hospital computer, server, or other computing device 602) via a cloud computing infrastructure (604), and interpreted using a knowledge database (606) created by artificial intelligence (AI). The data allow a group of medical doctors to determine the most appropriate clinical treatment for the patient. However, there is a significant technological gap here. A majority of existing biosensors available for IoT-based medicine are limited to physical sensors measuring body temperature, blood pressure, electrocardiogram (ECG), and physical motion. These biosensors fail to provide precise information on the disease condition based on biomarker-guided molecular diagnostics. A highly innovative network-connected POC biosensor system is the key to enabling such a smart medical system.

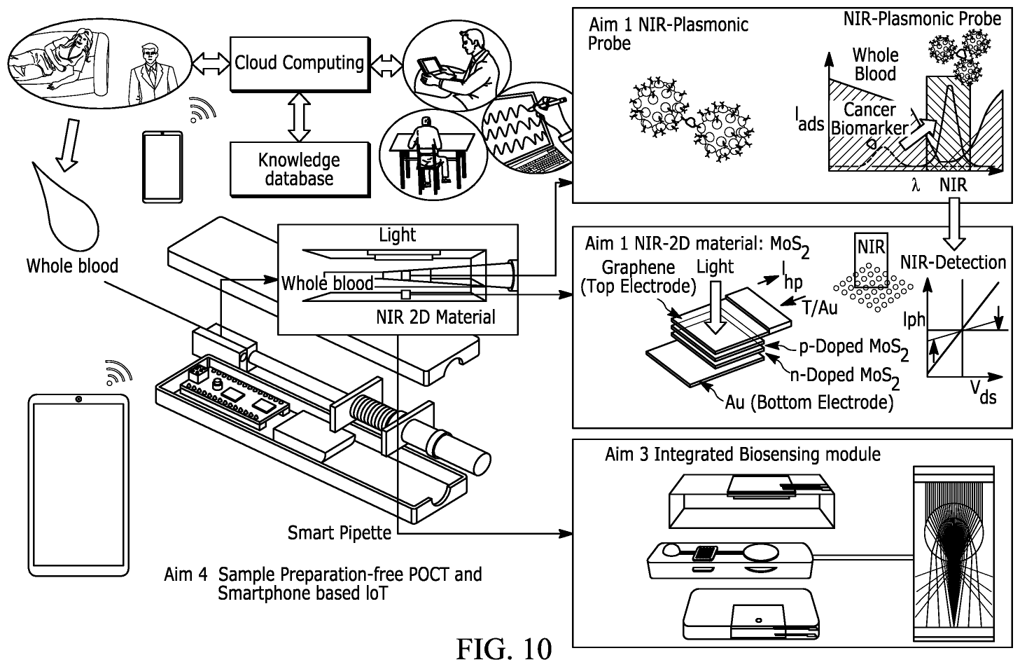

A biosensor-enabled smart diagnostic system connected to the IoT environment opens up incredible opportunities to realize early-stage treatment or prevention of serious conditions caused by cancer-related diseases. A highly portable POC biosensor module enabling ultrasensitive label-free on-chip colorimetric detection of cancer blood biomarkers with no laborious sample preparation/assay procedures could is provided in various examples herein. The POC biosensor module, e.g., the pipette 100, may be adopted for IoT operation by wireless transmission of data to a mobile smartphone. A pipette design is employed for the aimed module so that the system permits easy manual sample loading and manipulation for biomarker detection. As shown in FIG. 10, the entire smart pipette module may be equipped with a comprehensive set of functions leading to self-contained device operation. As discussed in examples herein, these functions include (1) microfluidic sample/reagent manipulation, (2) plasmonic label-free cancer biomarker protein sensing in whole blood, and (3) optoelectronic signal transduction in a near-infrared (NIR) optical band. The signal transduction in smart pipette exploits the extremely low-noise operation of photodetectors achieved by the electronic band structure of photoconductive 2D-layered materials. The 2D material-based photodetectors will be constructed using our in-house manufacturing techniques. Blood cells, platelets, and other liquid components of blood are nearly transparent in the NIR optical spectrum. Eliminating optical interferences by blood constituents, the NIR operation will allow Smart Pipette to achieve sample preparation-free, plasmonics-based label-free colorimetric POC cancer biomarker detection with high sensitivity, user-friendliness/accessibility, ease, and speed. The POC system, Smart Pipette, achieves unprecedentedly high performances.

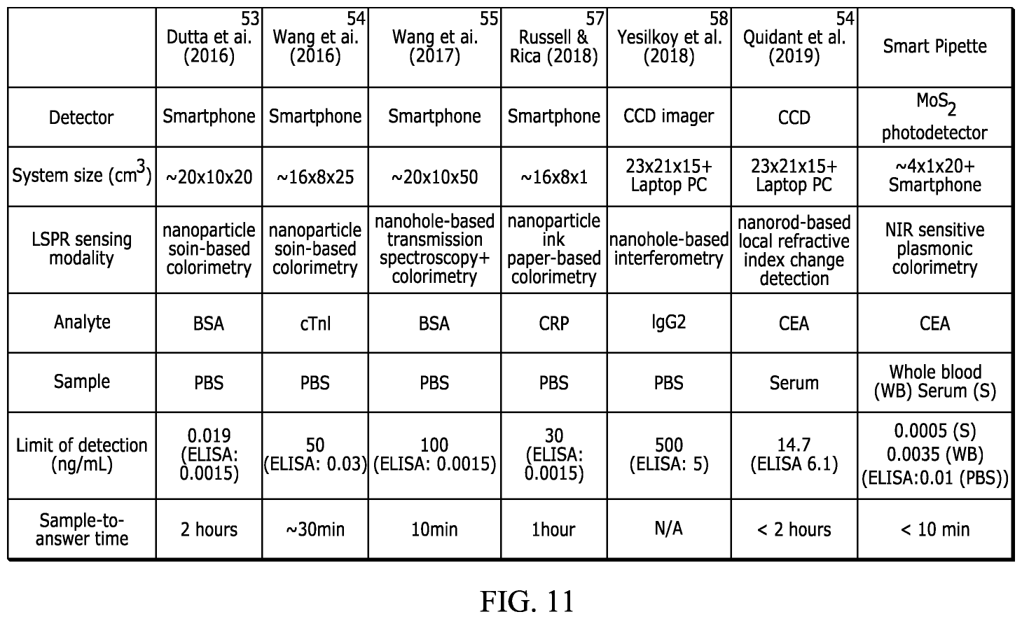

Over the last decade, many studies have developed LSPR biosensors for POC testing. A truly portable and self-contained system needs to incorporate several key components, including LSPR biosensing nanostructures, a fluidic system for sample handling, optical components, a light source, a photodetector for signal reading, and a photosignal processing electronic unit. FIG. 11 summarizes state-of-the-art LSPR-based POC immunosensor systems found in the literature. The most common detector used for these systems is a smartphone. Because smartphones contain a built-in light emitting diode (LED) flashlight source, a CMOS (complementary metal oxide semiconductor) imager, and embedded central processing and graphics processing units, they facilitate rapid, user-friendly analyte detection and wireless data transmission. Although the existing POC immune biosensor systems show some promise (some of them with an impressively short assay time), their implementations in real clinical settings have been seriously limited due to their poor sensitivity that prevents biomarker detection at the clinical threshold <3 ng/mL for cancer biomarkers. Previous work largely has focused on proof-of-concept prototyping. None of the devices has been used for multiplex analysis of cancer-relevant blood biomarkers in a POCT setting. Furthermore, the optical signal detection using a phone camera suffers a relatively low signal-to-noise ratio resulting from a high level of background noise in CMOS image sensors (unlike the layered 2D material-based detection proposed here). Hence, the existing state-of-the-art, portable POC devices unquestionably have much lower sensitivity than the gold standard ELISA. In contrast, the aimed device is to achieve nearly 3 orders of magnitude higher sensitivity than these POC devices.

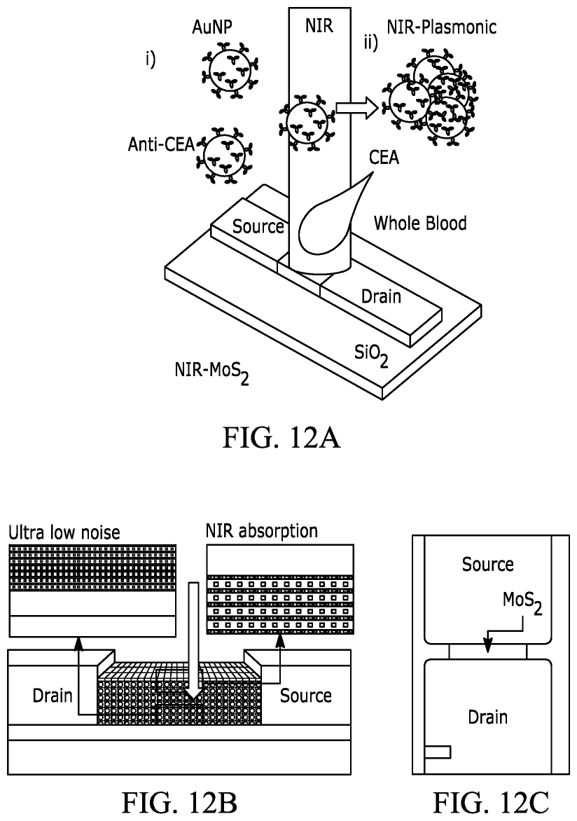

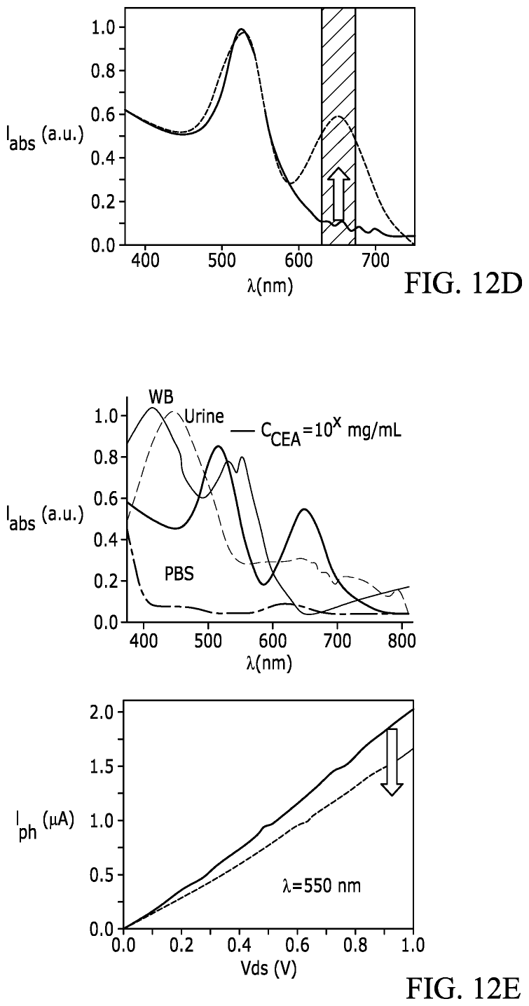

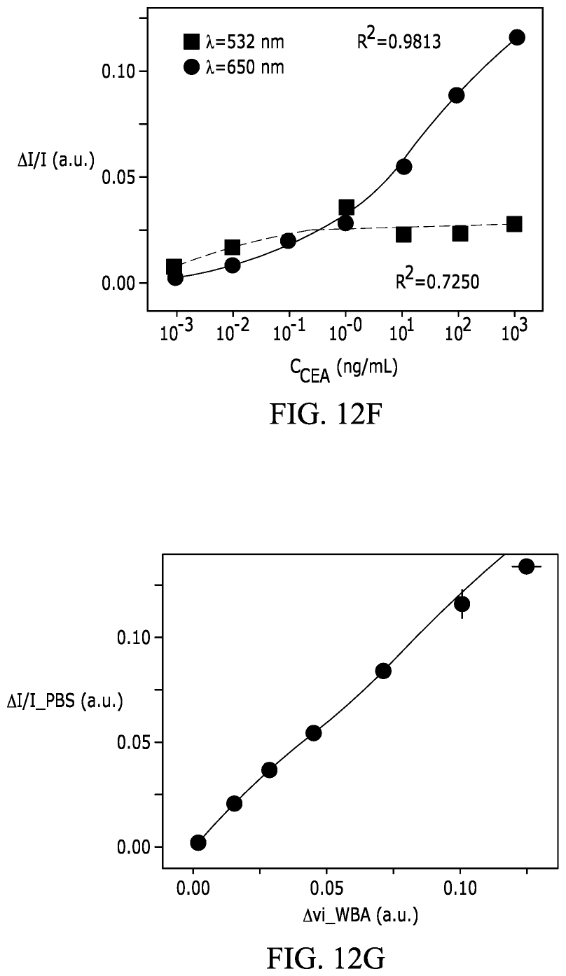

Instead, FIG. 12 illustrates an example biomarker detection strategy in accordance with the present techniques and in particular one that does not require plasma/serum isolation from whole blood, sample washing, or labeling so called “near-infrared (NIR) plasmonic-optoelectronic biosensensing.” In the illustrated example, the biosensor device achieves on-chip colorimetric measurement by integrating a microfluidic chamber that suspends plasmonic gold nanoparticles (AuNPs) in a biofluid and an ultra-low noise NIR-2D MoS₂ detector into a single chip, as shown in FIG. 12A. The AuNPs are coated with antibodies targeting specific biomarker protein molecules. The presence of the analytes causes the aggregation of these AuNPs, which leads to strong plasmonic coupling of incident light in the AuNP assembly due to the LSPR effect. An analyte-induced LSPR shift gives rise to a change in the absorbance spectrum of the solution suspending the AuNPs. The ultra-low noise photoconduction characteristic of the NIR-MoS₂ detector derives from its single-crystalline few-layer drain-source channel structure that absorbs a sufficient amount of NIR incident light and suppresses boundary scatterings of photo-excited electrons, FIG. 12B.

With such a device, the detection of cancer embryonic antigen (CEA) is possible in whole blood. As mentioned above, the CEA level (C_{CEA}) is found to be elevated in many cancer-related diseases, which allows CEA to serve as the first analyte in cancer-screening blood tests. A previous study reports that detection of low C_{CEA} in biofluids allows for monitoring early stages of cancer. When a biofluid sample containing CEA was loaded into the micro-chamber, the binding between CEA and anti-CEA-coated AuNPs increased the absorbance of incident light at λ=650 nm. The increased light absorbance decreased the photoconduction in the NIR-MoS₂ channel beneath the micro-chamber of the device.

In some examples, the length of the 2D MoS₂ channel can be as short as 1 μm, FIG. 12C. This keeps any grains from being formed within the channel. The AuNP size may be carefully selected to maximize the absorbance of the NIR optical band in the sample biofluid that accompanies analyte-induced nanoparticle aggregation, FIG. 12D. The absorbance spectrum measurement for various biofluids (whole blood, urine, and phosphate buffered saline (PBS)) confirms that whole blood is most transparent to light in the NIR region around λ=650 nm, FIG. 12E. Notably, the NIR operation is expected to minimize the influence of background constituents of whole blood on our biomarker analysis. The photocurrent (Iₚₕ) measured for the NIR-MoS₂ channel reveals the distinct change in its photoconduction upon loading a CEA sample at 10₋₃ ng/mL, FIG. 12E. Standard curves show the correlation between the C_{CEA} and the change in Iₚₕ for CEAspiked whole blood with incident lights at λ=532 and 650 nm. The standard curve at λ=650 nm shows excellent sensitivity to the change in C_{CEA} compared to the one obtained at λ=532 nm, where the light absorbance of whole blood becomes significantly high, FIG. 12F. The same measurement for background-free CEA-spiked purified PBS was performed and an excellent agreement with the whole blood test was confirmed, FIG. 12G. Thus, the NIR plasmonic-optoelectronic biosensing strategy experiences negligible interferences from the background of whole blood, permitting direct measurement of whole blood biomarkers without any sample preparation to isolate plasma or sample washing steps. The sample preparation free, wash-free, label-free biosensing measurement achieved “ultra-high” sensitivity (Limit of Detection=0.0035 ng/mL) within 10 min with a hundred-fold increase in the colorimetric signal, as compared with Enzyme-Linked Immunosorbent Assays (ELISA). This novel biosensing technology provides a strong foundation for the aimed ultra-sensitive, rapid POC platform, Smart Pipette.

In an example, the process involves mixing antibody-coated gold nanoparticles (AuNPs) suspended in a buffer solution with whole blood, loading the mixture to the device, incubating the mixture, where aggregation of the AuNPs gets induced by the presence of the analyte proteins in the whole blood, and detecting the near-infrared optical transmission change through the mixture due to the AuNP aggregation using the underlying photodetector. The near-infrared operation allows the photodetector to detect the signal change without any interference from the blood background (blood cells, platelets, and etc.). This leads to the label-free, wash-free analysis requiring any assay steps of sample processing, purification, modification, and washing.

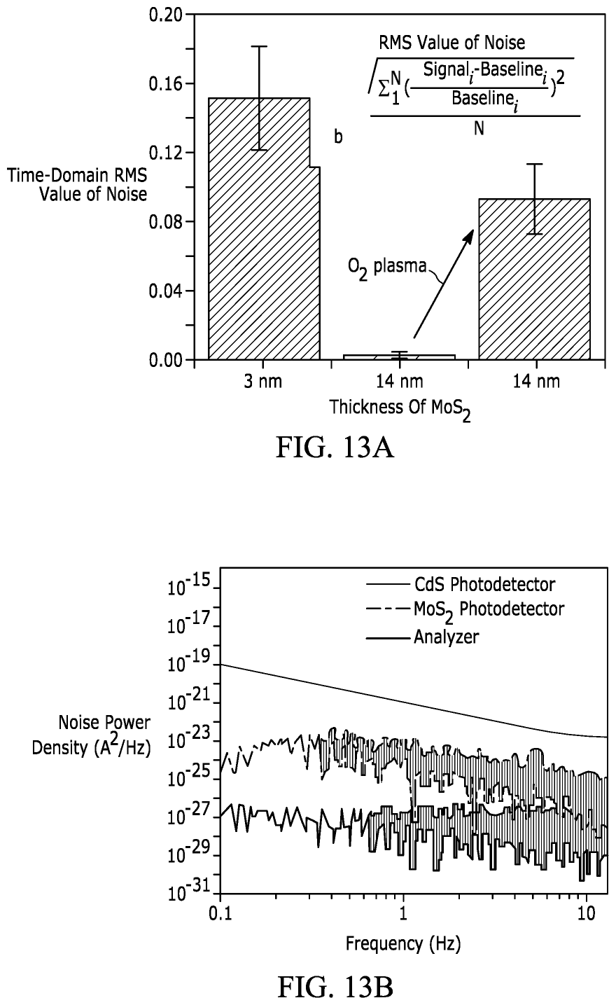

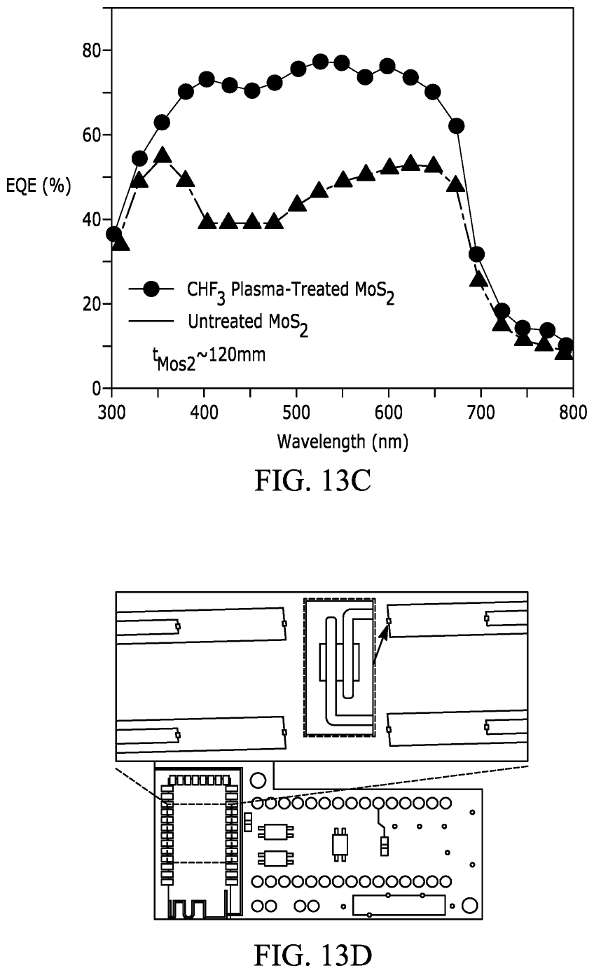

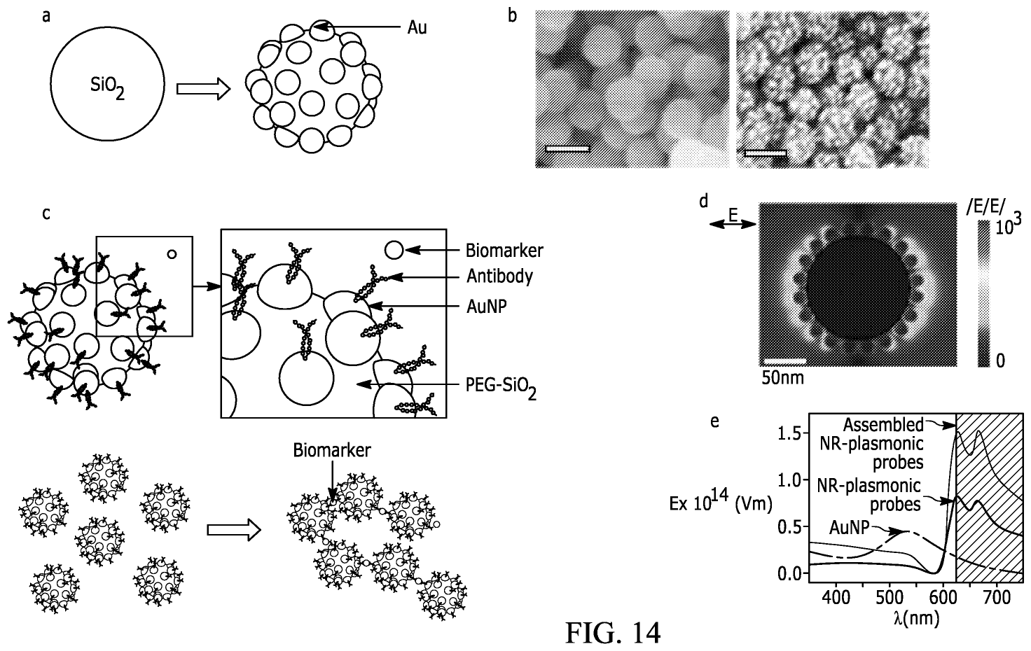

FIGS. 13A-13C illustrate the preliminary results of the study. FIG. 13A plots the RMS noise values measured from a 3 nm thick MoS₂ photodetector and a 14 nm thick device. Both devices are made from pristine few-layer MoS₂ and work in a photoconductor mode. This study shows that the thicker MoS₂ photoconductive layer exhibits a much lower noise level in comparison with the thinner one. This observation suggests that a thicker photoconductive layer could more effectively screen the potential disorder from the material interfaces, therefore leading to fewer scattering events for photogenerated carriers, in comparison with the thinner ones. FIG. 13A also shows that a brief O₂ plasma treatment prominently increases the noise level in the 14 nm thick device (but still lower than that measured from the 3 nm thick one). This further implies that a device fabrication route with the minimal usage of plasma processes (e.g., building of the photodetectors based on TMDC heterostructures, as proposed above) is highly desirable for achieving ultralow noise optoelectronic biosensors. After such a device analysis and optimization step in terms of noise level, the current MoS₂ photoconductive detectors exhibit an average noise power density about four orders of magnitude lower than that measured from commercial CdS photodetectors, as shown in FIG. 13B. FIG. 13C shows the external quantum efficiency (EQE) spectra measured from a plasma-doped MoS₂ photodetector and an undoped control device (both under zero external bias). The plasma-doped MoS₂ photodetector exhibits a very high EQE of ˜70% at around 650 nm (i.e., the operation wavelength for the proposed smart pipette system). The developed 2D photodetectors were integrated onto a PCB for wirelessly transmitting biosensing data (FIG. 13D).

This process involves controlling the thickness of the MoS₂ photodetector layer to the optical thickness of 14 nm results in the ultralow-noise photoconductive characteristic of the device, allowing achievement of the very high-sensitivity analyte measurement.

FIGS. 14A-14E illustrate NIR sensitive plasmonic nanoprobes that may be used to for colorimetric cancer biomarker detection, for example in the example detector of FIG. 12A. FIG. 14A illustrates a schematic of the NIR sensitive plasmonic nanoprobes. FIG. 14B illustrates a scanning electron microscope (SEM) images of SiO₂ particles and NIR sensitive plasmonic nanoprobes, and large-scale production (scale bar=50 nm). FIG. 14C illustrates NIR sensitive plasmonic nanoprobe aggregation driven by antigen biomarker-antibody binding events to be able to form a biomarker detector. FIG. 14D illustrates electric (E)-field distribution near the plasmonic nanoprobe surface. FIG. 14E illustrates an E-field intensity spectrum around an AuNS, a NIR sensitive nanoprobe, and aggregated nanoprobe particles.

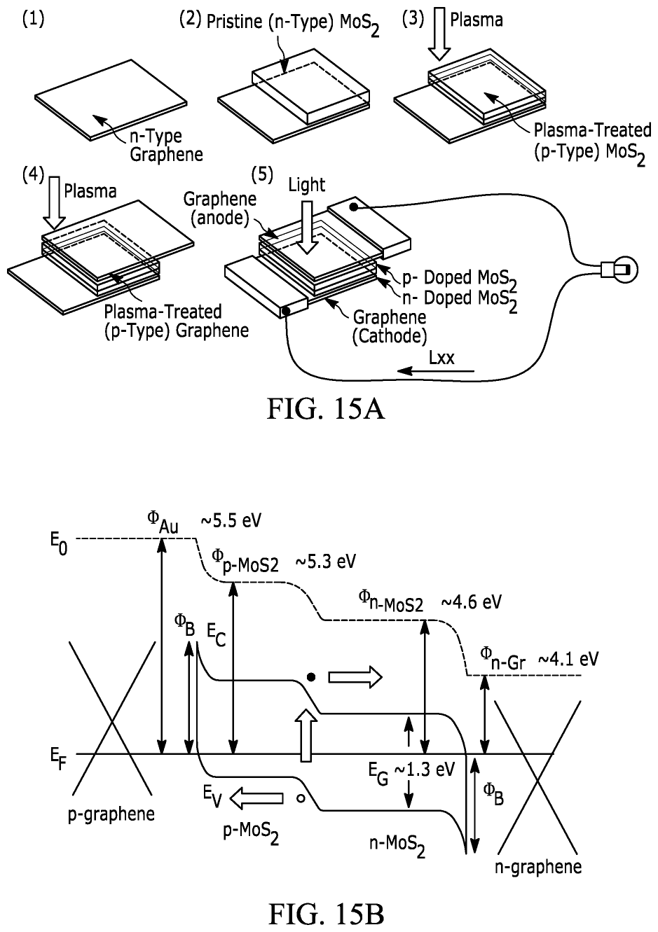

FIG. 15A illustrates an example fabrication method for an example photodetector in accordance with the configurations herein, including the configuration of FIG. 12A. FIG. 15B illustrates a band diagram of a photodetector with pGr/pM/nM/nGr heterostructure. In the example fabrication method, an n-Type graphene substrate is provided, at a first step, and an n-Type MoS₂ detection layer is formed on the graphene substrate over a detector region leaving an electrode receiving region. At a third step, a graphene second detection layer formed of p-Type MoS₂ is formed next, and at a fourth step, a p-Type graphene capping substrate is applied adjacent the two layer MoS₂ detector and leaving an electrode receiving region. As shown, the two electrode receiving regions are capped with a deposited electrode for photodetector signal detection.

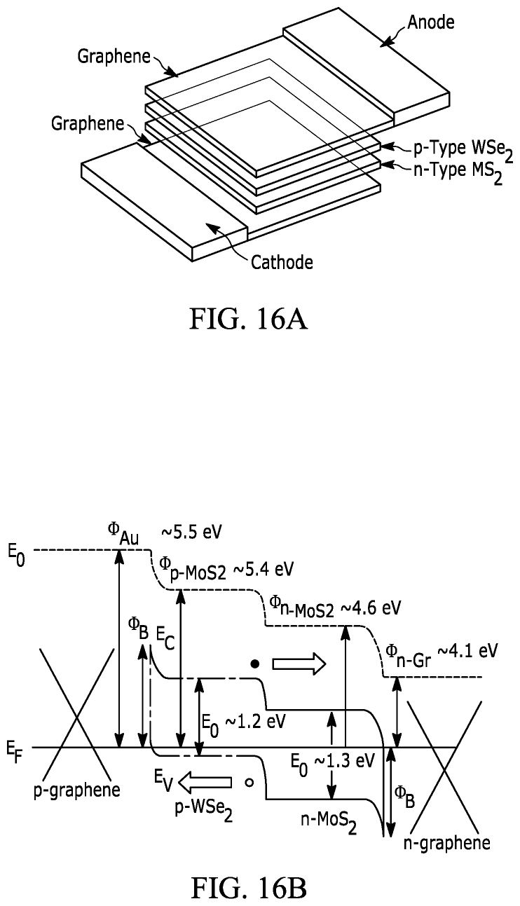

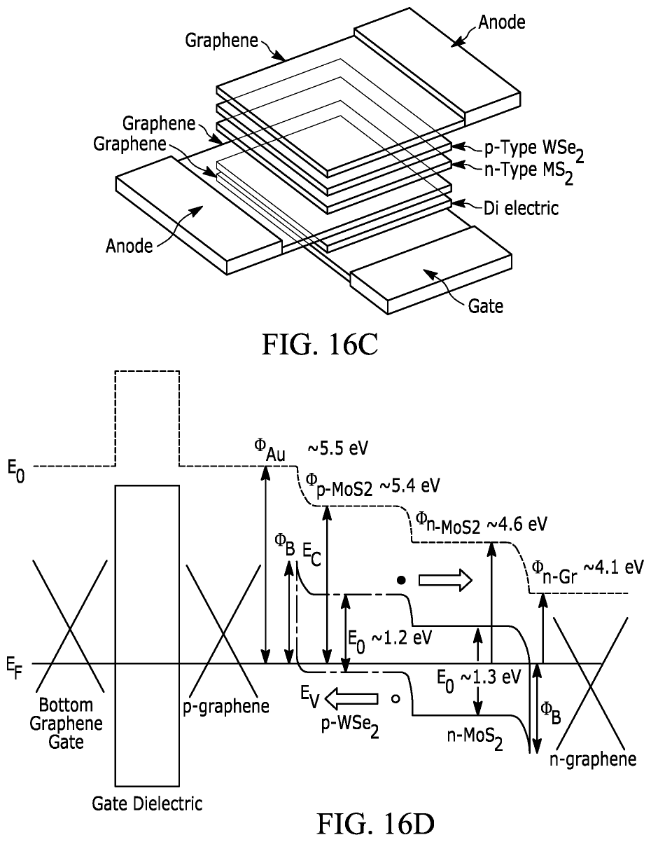

FIG. 16A illustrates an example fabricated photodetector in accordance with configurations herein, including the configuration of FIG. 12A, having a pGr/pW/nM/nGr heterostructure. FIG. 16B illustrates a band diagram of a photodetector with pGr/pW/nM/nGr heterostructure. In the illustrated example, WSe₂ and MoS₂ form a type-II heterojunction at their interface. FIG. 16C illustrates an example fabricated photodetector similar to that of FIG. 16A including a dielectric and having an electrically controllable gate electrode, for example, formed on a second electrode receiving region of a graphene substrate. FIG. 16D illustrates a band diagram of a gated 2D heterostructure.

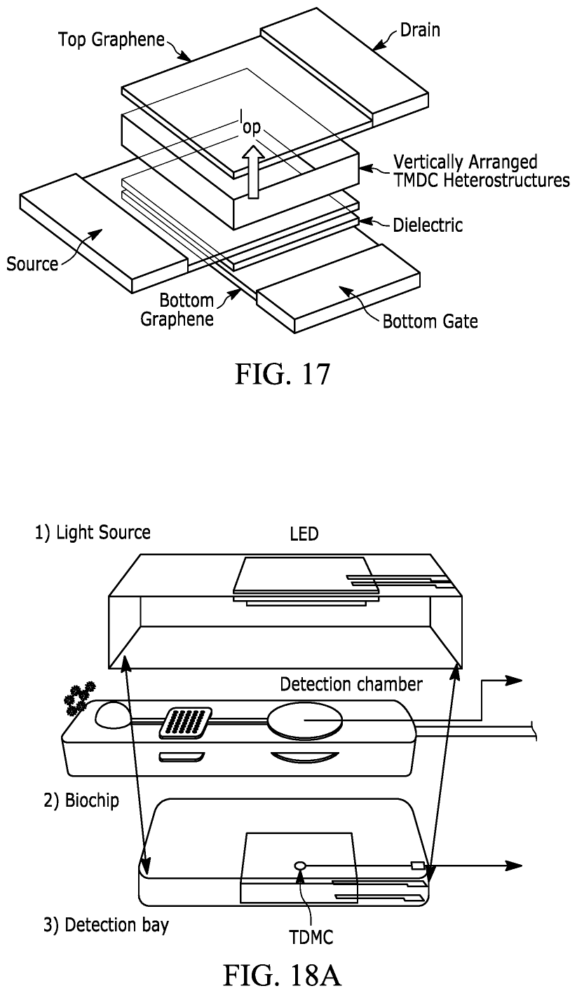

FIG. 17 illustrates an another example photodetector configuration similar to that of FIG. 16C but including a vertically aligned NIR-Transition metal dichalcogenide monolayers (TMDC) photodetector heterostructure.

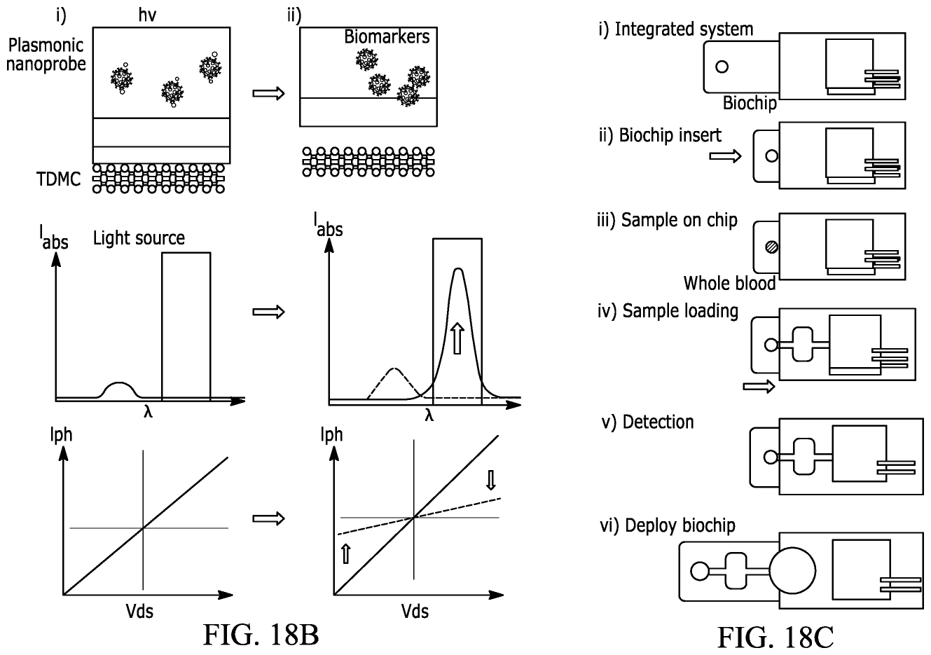

FIG. 18A illustrates another example integrated system biochip in exploded form and having a biomarker detector in the form of a handheld module assembled by inserting layers of a LED chip, a microfluidic biochip with a plasma isolation filter and a sample detection chamber, and a TMDC photodetector into a cartridge-spacer body frame, in accordance with an example. FIG. 18B illustrates a NIR colorimetry detection principle. FIG. 18C illustrates detection steps. A biosensor chip, such as those described in examples herein, is inserted into an integrated system housing having a sample region for positioning a blood sample. The sample material may transition to a detection region through a sample loading process, such as capillary movement of the sample along a fluid path. Upon detection, the sample material may transition to a detection chamber that is aligned with the LED light source and NIR-TMDC photodetector.

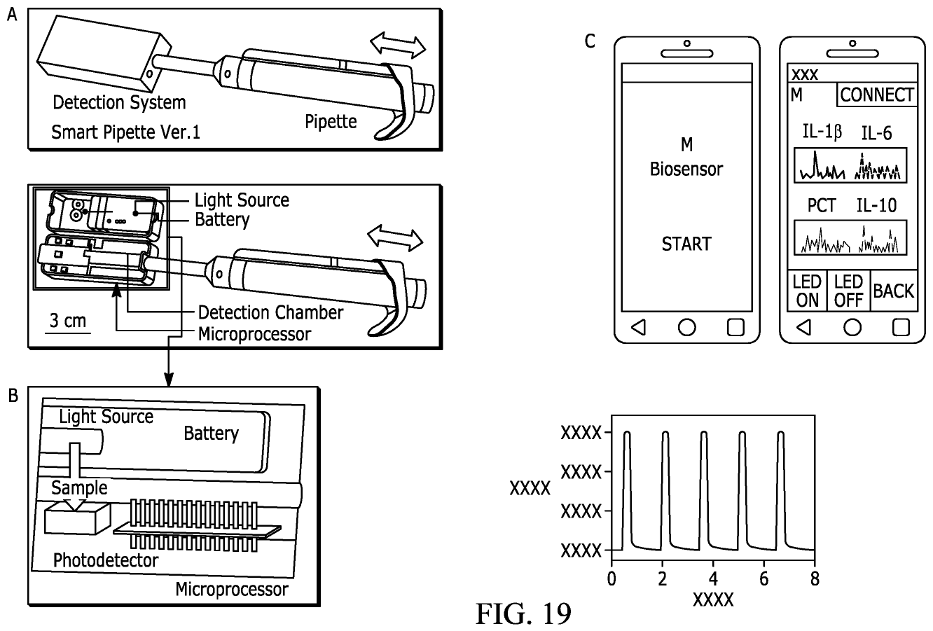

FIG. 19A illustrates a pipette, in accordance with another example. FIG. 19B illustrates a micro-optics module (μOM) for software testing using a mobile device, such as a mobile phone. The uOM module incorporates off-the-shelf commercial photodetector (with limited sensitivity) and LED components. FIG. 19C illustrates screenshots of generate biomarker detection plots presented by one or more apps executed on a mobile computing device, such as smartphone to provide smartphone-based data access.

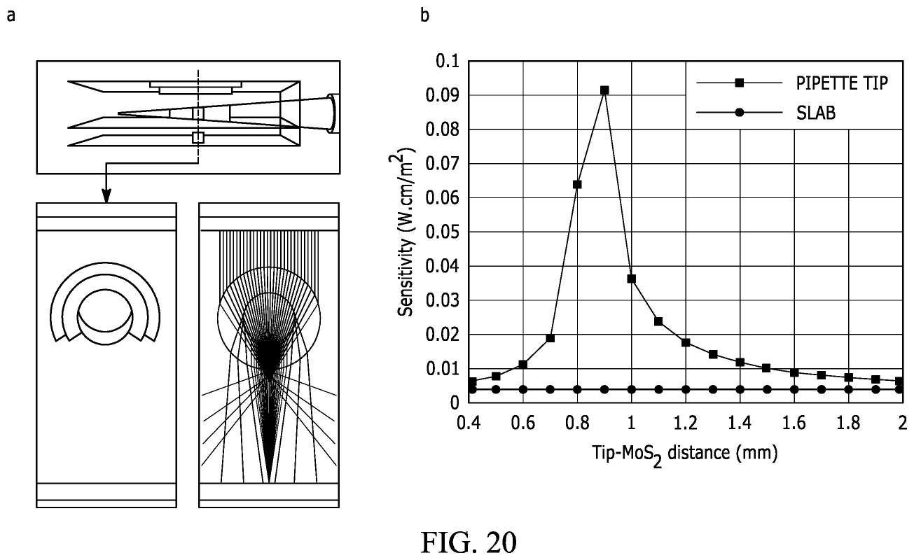

FIGS. 20A & 20B illustrate a pipette biosensing module, in accordance with an example. FIG. 20A illustrates geometric optics simulation results showing that micro-lens like biochip flow-cell structure leading to signal amplification. FIG. 20B illustrates a calculated sensitivity indicating that the micro-lens like biochip structure would enable 20 times more sensitive detection than a conventional slab channel structure.

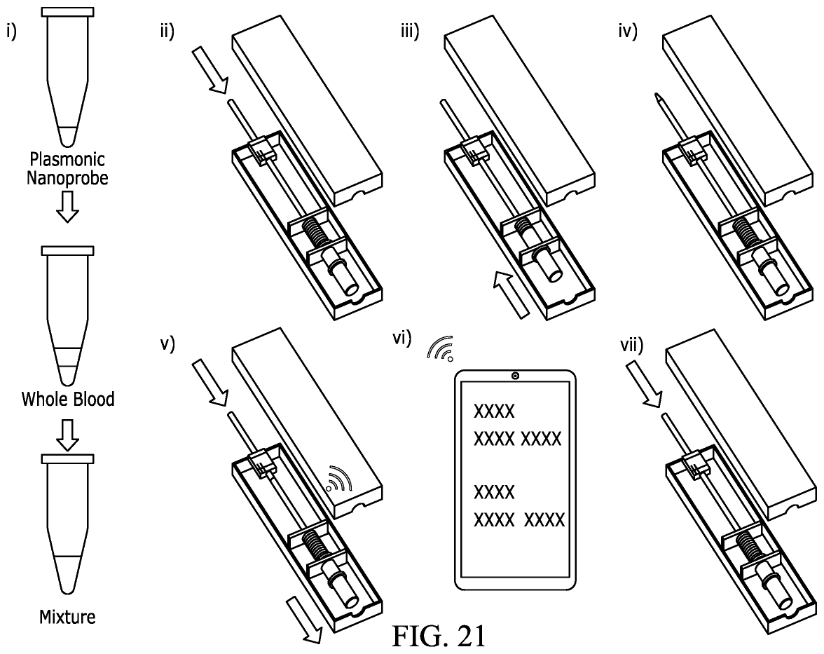

FIG. 21 illustrates a portable biomarker analysis module, in accordance with an example showing different steps of operation. In FIG. 21, step i) illustrates mixing nanoprobes and whole blood in a sample tube. Step ii) illustrates inserting the biochip tip into the sample tube. Step iii) illustrates pushing the pipette plunger. Step iv) illustrates releasing the plunger to load the sample into the biochip. Step v) illustrates incubating the sample and detecting the target biomarker. Step vi) illustrates transmitting the data to a smartphone or other mobile computing device. Step vii) illustrates replacing the used biochip with new one.

### ADDITIONAL ASPECTS

Aspect 1. A biomarker detector device comprising:
a sealable housing;
an inlet configured to receive fluid; and
a sensor device within the sealable housing and communicatively coupled to the inlet to receive the fluid, the sensor further comprising an illumination source, a photodetector array, and a microfluidic chip positioned between the illumination source and the photodetector array, the microfluidic chip comprising a plurality of barcode channels, each barcode channel configured to detect to a different biomarker and each barcode channel configured to affect illumination from the illumination source in response to detection of the respective biomarker, where such affected illumination is detectable by the photodetector array.

Aspect 2. The biomarker detector device of aspect 1, wherein the microfluidic chip is a nanoplasmonic barcode chip having a plurality of different nanoplasmonic barcode detectors.

Aspect 3. The biomarker detector device of aspect 1, wherein each of the barcode channels comprises a different antibody each different antibody selected to capture a different biomarker.

Aspect 4. The biomarker detector device of aspect 3, wherein each barcode channel antibody has an antibody selected to capture one of cytokines, complement system biomarkers, sepsis biomarkers, lung-injury biomarkers, brain-injury biomarkers, and metabolites.

Aspect 5. The biomarker detector device of aspect 3, wherein each barcode channel antibody has an antibody selected to capture one of IL-2, INF-γ, IL-4, IL-10, IL-1β, TNF-α, IL-6, C3a, C5a, C5b-9, Bb, C4d, CRP PCT, CitH3, SPD, sRAGE, KL-6, CC-16, PAI-1, protein C, vWF, HMGB1, NSE, S100β, GFAP, UCHL1, BDNF, NFL, D-lactate, and histamine.

Aspect 6. The biomarker detector device of aspect 1, wherein each barcode channel has an antibody selected to capture a cancer biomarker selected from the group consisting of carcinoembryonic antigen (CEA), cancer antigen (CA)19-9, prostate-specific antigen (PSA), human epidermal growth factor receptor HER2, AFP, (CA)125, HE4, OVA1 test, ROMA test, (CA)15-2, (CA)27-29, Tg, and hCG.

Aspect 7. The biomarker detector device of aspect 1, wherein each barcode channel has an antibody selected to capture a SARS-Cov spike protein or a SARS-Cov-2 spike protein.

Aspect 8. The biomarker detector device of aspect 1, further comprising a plasma separation chamber configured to separate the fluid into plasma and non-plasma, wherein the plasma separation chamber is configured to send the plasma to the microfluidic chip, and wherein the microfluidic chip is a nanoplasmonic barcode chip having a plurality of nanoplasmonic barcode detectors.

Aspect 9. The biomarker detector device of aspect 1, wherein the photodetector array is MoS₂ photodetector channel array.

Aspect 10. The biomarker detector device of aspect 1, wherein the illumination source is an organic light emitting diode (OLED) source.

Aspect 11. The biomarker detector device of aspect 1, wherein each barcode channel is configured as a localized surface plasmon resonance (LSPR) nanoplasmonic biosensor, and wherein the each LSPR nanoplasmonic biosensor is equally spaced from at least one other LSPR nanoplasmonic biosensor.

Aspect 12. The biomarker detector device of aspect 1, further comprising a plunger assembly engaged with the sensor and actionable to draw fluid into the inlet upon engagement of a push button of the plunger assembly, wherein a spring activation of the plunger assembly is housed with the housing.

Aspect 13. The biomarker detector device of aspect 1, further comprising a wireless data transmitter within the housing and configured to wirelessly transmit one or more detection signals from the photodetector array and corresponding to one or more different biomarkers detected by one or more of the barcode channels.

Aspect 14. A biomarker analysis system comprising the biomarker detector device of aspect 9 and further comprising:
a mobile computing device external to and configured to wirelessly communication with the biomarker detector device, the mobile computing device having a display and a wireless data transmitter, the mobile computing communicatively coupled to receive biomarker data from the biomarker detector device over a wireless communication link, the mobile computing device having a processor and a memory storing instructions that when executed cause the processor to generate a biomarker report indicating a presence of one or more biomarkers detected by the biomarker detector device and to display the biomarker report on the display of the mobile computing device.

Aspect 15. The biomarker analysis system of aspect 14, wherein the mobile computing device is a smartphone.

Aspect 16. The biomarker analysis system of aspect 14, further comprising a machine learning framework configured to classify biomarker data based on severity of illness or injury.

Aspect 17. The biomarker analysis system of aspect 14, further comprising a machine learning framework configured to classify biomarker data based on severity of sepsis.

Aspect 18. The biomarker analysis system of aspect 14, further comprising a machine learning framework configured to classify biomarker data based on severity of lung injury.

Aspect 19. The biomarker analysis system of aspect 14, further comprising a machine learning framework configured to classify biomarker data based on severity of brain injury.

Aspect 20. A biomarker detector device comprising:
a sealable housing;
an inlet configured to receive fluid; and
a biosensor assembly within the sealable housing and communicatively coupled to the inlet to receive the fluid, the biosensor assembly being a multilayered structure comprising an illumination source in a first layer, a photodetector array in a second layer opposing the first layer, and a biosensor in an intermediate layer positioned between the first layer and the second layer, the biosensor being configured with at least one near infrared (NIR) sensitive plasmonic nanoprobe for colorimetric cancer biomarker detection by affecting illumination from the illumination source, where such affected illumination is detectable by the photodetector array.

Aspect 21. The biomarker detector device of aspect 20, wherein the at least one NIR sensitive plasmonic nanoprobe comprises an integrated a microfluidic chamber that suspends plasmonic gold nanoparticles (AuNPs) in a biofluid for the plasmonic nanoprobe and the photodetector is a NIR-two dimensional (2D) MoS₂ photodetector operatively positioned to receive the affected illumination from the microfluidic chamber.

Aspect 22. The biomarker detector device of aspect 21, wherein the AuNPs are coated with at least one antibody for targeting a cancer specific biomarker selected from the group consisting of carcinoembryonic antigen (CEA), cancer antigen (CA)19-9, prostate-specific antigen (PSA), human epidermal growth factor receptor HER2, AFP, (CA)125, HE4, OVA1 test, ROMA test, (CA)15-2, (CA)27-29, Tg, and hCG.

Aspect 23. The biomarker detector device of aspect 21, wherein the NIR-2D MoS₂ photodetector is a multiple layer structure having a drain and source control structure.

Aspect 24. The biomarker detector device of aspect 23, wherein the multiple layer structure comprises, in layer order, a n-Type graphene layer forming a cathode, a N-Type MoS₂ layer, a p-Type MoS₂ layer, and a p-Type graphene layer forming an anode.

Aspect 25. The biomarker detector device of aspect 23, wherein the multiple layer structure comprises, in layer order, a gate electrode graphene layer, a dielectric layer, a cathode graphene layer, a N-Type MoS₂ layer, a p-Type MoS₂ layer, and an anode graphene layer.

Aspect 26. The biomarker detector device of aspect 23, wherein the multiple layer structure comprises, in layer order, a gate electrode graphene layer, a dielectric layer, a cathode graphene layer, a NIR-Transition metal dichalcogenide monolayers (TMDC) photodetector heterostructure, and an anode graphene layer.

Aspect 27. A biomarker detector comprising:
a sealable housing;
an inlet configured to receive fluid; and
a sensor device within the sealable housing and communicatively coupled to the inlet to receive the fluid, the sensor further comprising an illumination source, a photodetector array, and a microfluidic chip positioned between the illumination source and the photodetector array, the microfluidic chip comprising a microfluidic chamber holding a sample/colloidal nanoparticle biosensor mixture solution to permit colorimetric analysis of analyte-induced nanoparticle aggregation while it is aligned with the illumination source and the photodetector array in the biomarker detector architecture.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

What is claimed:

1. A biomarker detector device comprising:
a sealable housing;
an inlet configured to receive fluid; and
a sensor device within the sealable housing and communicatively coupled to the inlet to receive the fluid, the sensor further comprising an illumination source, a photodetector array, and a microfluidic chip positioned between the illumination source and the photodetector array, the microfluidic chip comprising a plurality of barcode channels, each barcode channel configured to detect to a different biomarker and each barcode channel configured to affect illumination from the illumination source in response to detection of the respective biomarker, where such affected illumination is detectable by the photodetector array.

2. The biomarker detector device of claim 1, wherein the microfluidic chip is a nanoplasmonic barcode chip having a plurality of different nanoplasmonic barcode detectors.

3. The biomarker detector device of claim 1, wherein each of the barcode channels comprises a different antibody each different antibody selected to capture a different biomarker.

4. The biomarker detector device of claim 3, wherein each barcode channel antibody has an antibody selected to capture one of cytokines, complement system biomarkers, sepsis biomarkers, lung-injury biomarkers, brain-injury biomarkers, and metabolites.

5. The biomarker detector device of claim 3, wherein each barcode channel antibody has an antibody selected to capture one of IL-2, INF-γ, IL-4, IL-10, IL-1β, TNF-α, IL-6, C3a, C5a, C5b-9, Bb, C4d, CRP PCT, CitH3, SPD, sRAGE, KL-6, CC-16, PAI-1, protein C, vWF, HMGB1, NSE, S100β, GFAP, UCHL1, BDNF, NFL, D-lactate, and histamine.

6. The biomarker detector device of claim 1, wherein each barcode channel has an antibody selected to capture a cancer biomarker selected from the group consisting of carcinoembryonic antigen (CEA), cancer antigen (CA)19-9, prostate-specific antigen (PSA), human epidermal growth factor receptor HER2, AFP, (CA)125, HE4, OVA1 test, ROMA test, (CA)15-2, (CA)27-29, Tg, and hCG.

7. The biomarker detector device of claim 1, wherein each barcode channel has an antibody selected to capture a SARS-Cov spike protein or a SARS-Cov-2 spike protein.

8. The biomarker detector device of claim 1, further comprising a plasma separation chamber configured to separate the fluid into plasma and non-plasma, wherein the plasma separation chamber is configured to send the plasma to the microfluidic chip, and wherein the microfluidic chip is a nanoplasmonic barcode chip having a plurality of nanoplasmonic barcode detectors.

9. The biomarker detector device of claim 1, wherein the photodetector array is MoS₂ photodetector channel array.

10. The biomarker detector device of claim 1, wherein the illumination source is an organic light emitting diode (OLED) source.

11. The biomarker detector device of claim 1, wherein each barcode channel is configured as a localized surface plasmon resonance (LSPR) nanoplasmonic biosensor, and wherein the each LSPR nanoplasmonic biosensor is equally spaced from at least one other LSPR nanoplasmonic biosensor.

12. The biomarker detector device of claim 1, further comprising a plunger assembly engaged with the sensor and actionable to draw fluid into the inlet upon engagement of a push button of the plunger assembly, wherein a spring activation of the plunger assembly is housed with the housing.

13. The biomarker detector device of claim 1, further comprising a wireless data transmitter within the housing and configured to wirelessly transmit one or more detection signals from the photodetector array and corresponding to one or more different biomarkers detected by one or more of the barcode channels.

14. A biomarker analysis system comprising the biomarker detector device of claim 9 and further comprising:
a mobile computing device external to and configured to wirelessly communication with the biomarker detector device, the mobile computing device having a display and a wireless data transmitter, the mobile computing communicatively coupled to receive biomarker data from the biomarker detector device over a wireless communication link, the mobile computing device having a processor and a memory storing instructions that when executed cause the processor to generate a biomarker report indicating a presence of one or more biomarkers detected by the biomarker detector device and to display the biomarker report on the display of the mobile computing device.

15. The biomarker analysis system of claim 14, wherein the mobile computing device is a smartphone.

16. The biomarker analysis system of claim 14, further comprising a machine learning framework configured to classify biomarker data based on severity of illness or injury.

17. The biomarker analysis system of claim 14, further comprising a machine learning framework configured to classify biomarker data based on severity of sepsis.

18. The biomarker analysis system of claim 14, further comprising a machine learning framework configured to classify biomarker data based on severity of lung injury.

19. The biomarker analysis system of claim 14, further comprising a machine learning framework configured to classify biomarker data based on severity of brain injury.

20. A biomarker detector device comprising:
a sealable housing;
an inlet configured to receive fluid; and
a biosensor assembly within the sealable housing and communicatively coupled to the inlet to receive the fluid, the biosensor assembly being a multilayered structure comprising an illumination source in a first layer, a photodetector array in a second layer opposing the first layer, and a biosensor in an intermediate layer positioned between the first layer and the second layer, the biosensor being configured with at least one near infrared (NIR) sensitive plasmonic nanoprobe for colorimetric cancer biomarker detection by affecting illumination from the illumination source, where such affected illumination is detectable by the photodetector array.

21. The biomarker detector device of claim 20, wherein the at least one NIR sensitive plasmonic nanoprobe comprises an integrated a microfluidic chamber that suspends plasmonic gold nanoparticles (AuNPs) in a biofluid for the plasmonic nanoprobe and the photodetector is a NIR-two dimensional (2D) MoS₂ photodetector operatively positioned to receive the affected illumination from the microfluidic chamber.

22. The biomarker detector device of claim 21, wherein the AuNPs are coated with at least one antibody for targeting a cancer specific biomarker selected from the group consisting of carcinoembryonic antigen (CEA), cancer antigen (CA)19-9, prostate-specific antigen (PSA), human epidermal growth factor receptor HER2, AFP, (CA)125, HE4, OVA1 test, ROMA test, (CA)15-2, (CA)27-29, Tg, and hCG.

23. The biomarker detector device of claim 21, wherein the NIR-2D MoS₂ photodetector is a multiple layer structure having a drain and source control structure.

24. The biomarker detector device of claim 23, wherein the multiple layer structure comprises, in layer order, a n-Type graphene layer forming a cathode, a N-Type MoS₂ layer, a p-Type MoS₂ layer, and a p-Type graphene layer forming an anode.

25. The biomarker detector device of claim 23, wherein the multiple layer structure comprises, in layer order, a gate electrode graphene layer, a dielectric layer, a cathode graphene layer, a N-Type MoS₂ layer, a p-Type MoS₂ layer, and an anode graphene layer.

26. The biomarker detector device of claim 23, wherein the multiple layer structure comprises, in layer order, a gate electrode graphene layer, a dielectric layer, a cathode graphene layer, a NIR-Transition metal dichalcogenide monolayers (TMDC) photodetector heterostructure, and an anode graphene layer.

27. A biomarker detector comprising:
a sealable housing;
an inlet configured to receive fluid; and
a sensor device within the sealable housing and communicatively coupled to the inlet to receive the fluid, the sensor further comprising an illumination source, a photodetector array, and a microfluidic chip positioned between the illumination source and the photodetector array, the microfluidic chip comprising a microfluidic chamber holding a sample/colloidal nanoparticle biosensor mixture solution to permit colorimetric analysis of analyte-induced nanoparticle aggregation while it is aligned with the illumination source and the photodetector array in the biomarker detector architecture.
